SEQUENCE LISTING (8 sequences)

SEQ ID NO: 1 - RNA, 19 nt, Artificial Sequence
  Description of Artificial Sequence  Synthetic Primer
cgauuccuucuaacagaaa

SEQ ID NO: 2 - RNA, 19 nt, Artificial Sequence
  Description of Artificial Sequence  Synthetic Primer
uuucuguuagaaggaaucg

SEQ ID NO: 3 - RNA, 19 nt, Artificial Sequence
  Description of Artificial Sequence  Synthetic Primer
gcgacguuccugaaaccac

SEQ ID NO: 4 - RNA, 19 nt, Artificial Sequence
  Description of Artificial Sequence  Synthetic Primer
gugguuucaggaacgucgc

SEQ ID NO: 5 - RNA, 19 nt, Artificial Sequence
  Description of Artificial Sequence  Synthetic Primer
ggucauccaugacaacuuu

SEQ ID NO: 6 - RNA, 19 nt, Artificial Sequence
  Description of Artificial Sequence  Synthetic Primer
aaaguugucauggaugacc

SEQ ID NO: 7 - RNA, 19 nt, Artificial Sequence
  Description of Artificial Sequence  Synthetic Primer
acuaccguuguuauaggug

SEQ ID NO: 8 - RNA, 19 nt, Artificial Sequence
  Description of Artificial Sequence  Synthetic Primer
ugauggcaacaauauccac

(19) United States
(12) Patent Application Publication
(10) Pub. No.: US 2004/0248094 A1
(43) Pub. Date: Dec. 9, 2004

(54) Methods and compositions relating to labeled RNA molecules that reduce gene expression

(76) Inventors: Lance P. Ford, Austin, TX (US); Mike Byrom, Austin, TX (US); Brittan L. Pasloske, Austin, TX (US)

Correspondence Address: Gina N. Shishima, FULBRIGHT & JAWORSKI L.L.P., Suite 2400, 600 Congress Avenue, Austin, TX, 78701, US

(21) Appl. No.: 10/360,772

(22) Filed: Jun. 12, 2002

(51) Int. Cl. C12Q 1/68; C07H 21/04

(52) U.S. Cl. 435/6; 536/25.32

(57) ABSTRACT

The present invention concerns methods and compositions involving labeled, double-stranded RNA (dsRNA), including siRNA, capable of triggering RNA-mediated interference (RNAi) in a cell. Compositions of the invention include labeled dsRNA for RNAi, which may be a single strand of RNA that basepairs with itself or two separate RNA strands. In some embodiments, the label is fluorescent. The present invention further concerns methods for preparing such composition and kits for implementing such methods. Other methods of the invention include ways of using labeled dsRNA for RNAi.

### BACKGROUND OF THE INVENTION

1. Field of the invention

The present invention relates generally to the field of molecular biology. More particularly, it concerns labeled, double-stranded RNA capable of reducing target gene expression in vitro and in vivo. These labeled interfering RNAs enable the analysis of their bio-distribution and metabolism and have applications in diagnostics and therapeutics.

2. Description of the Related Art

RNA interference (RNAi), originally discovered in Caenorhabditis elegans by Fire and Mello (Fire et al., 1998), is a phenomenon in which double stranded RNA (dsRNA) reduces the expression of the gene to which the dsRNA corresponds. The phenomenon of RNAi was subsequently proven to exist in many organisms and to be a naturally occurring cellular process. The RNAi pathway can be used by the organism to inhibit viral infections, transposon jumping and to regulate the expression of endogenous genes (Huntvagner et al., 2001; Tuschl, 2001; Waterhouse et al., 2001; Zamore 2001). In original studies, researchers were inducing RNAi in non-mammalian systems and were using long double stranded RNAs. However, most mammalian cells have a potent antiviral response causing global changes in gene expression patterns in response to long dsRNA thus arousing questions as to the existence of RNAi in humans. As more information about the mechanistic aspects of RNAi was gathered, RNAi in mammalian cells was shown to also exist.

In an in vitro system derived from Drosophila embryos long dsRNAs are processed into shorter small interfering (si) RNA the smaller siRNA by a cellular ribonuclease containing RNaseIII motifs (Bernstein et al., 2001; Grishok et al., 2001; Hamilton and Baulcombe, 1999; Knight and Bass, 2001; Zamore et al., 2000). Genetics studies done in C. elegans, N. crassa and A. thaliana have lead to the identification of additional components of the RNAi pathway. These genes include putative nucleases (Ketting et al., 1999), RNA-dependent RNA polymerases (Cogoni and Macino, 1999; Dalmay et al., 2000; Mourrain et al., 2000; Smardon et al., 2000) and helicases (Cogoni and Macino, 1999; Dalmay et al., 2001; Wu-Scharf et al., 2000). Several of these genes found in these functional screens are involved not only in RNAi but also in nonsense mediated mRNA decay, protection against transposon-transposition (Zamore, 2001), viral infection (Waterhouse et al., 2001), and embryonic development (Hutvagner et al., 2001; Knight and Bass, 2001). In general, it is thought that once the siRNAs are generated from longer dsRNAs in the cell by the RNaseIII like enzyme, the siRNA associate with a protein complex. The protein complex also called RNA-induced silencing complex (RISC), then guides the smaller 21 base double stranded siRNA to the mRNA where the two strands of the double stranded RNA separate, the antisense strand associates with the mRNA and a nuclease cleaves the mRNA at the site where the antisense strand of the siRNA binds (Hammond et al., 2001). The MRNA is then subsequently degraded by cellular nucleases.

Based upon some of the information mentioned above, Elbashir et al. (2001) discovered a clever method to bypass the anti viral response and induce gene specific silencing in mammalian cells. Several 21 nucleotide dsRNAs with 2 nucleotide 3′ overhangs were transfected into mammalian cells without inducing the antiviral response. The small dsRNA molecules (also referred to as “siRNA”) were capable of inducing the specific suppression of target genes. In one set of experiments, siRNAs complementary to the luciferase gene were co-transfected with a luciferase reporter plasmid into NIH3T3, COS-7, HeLaS3, and 293 cells. In all cases, the siRNAs were able to specifically reduce luciferase gene expression. In addition, the authors demonstrated that siRNAs could reduce the expression of several endogenous genes in human cells. The endogenous targets were lamin A/C, lamin B1, nuclear mitotic apparatus protein, and vimentin. The use of siRNAs to modulate gene expression has now been reproduced by at least two other labs (Caplen et al., 2001; Hutvagner et al., 2001) and has been shown to exist in more that 10 different organisms spanning a large spectrum of the evolutionary tree. RNAi in mammalian cells has the ability to rapidly expand our knowledge of gene function and cure and diagnose human diseases. However, much about the process is still unknown and thus, additional research and understanding will be required to take full advantage of it.

To gain insight into the implementation of molecules in a physiological system, such as nucleic acids, many labeling techniques have been employed to track drug discovery molecules in vitro and in vivo. The most popular methods of tagging molecules such as antisense compounds are with either fluorescent dyes or using radioactive molecules. Fluorescent dyes can be but are not limited to fluorescein, rhodomine, Oregon green, TAMRA and Texas Red (Regnier V, 1998; Yoo H, 2000; Zhang SP, 1996). Compounds that are y or positron emitters are also often used to study drugs in vivo and in vitro situations and can be but are not limited to 1231, technetium-99m, 111In, 125I, 3H, 35S, 18F, 76Br (Dewanjee et el., 1994; Kuhnast et al., 2000; Liu et al., 2001; Rusckowski et al., 2000; Sedelnikova et al., 2000; 2000; Wu, et al., 2000). Researchers are trying to get away from radioactive compounds since the radioactivity used often leads to DNA damage and the accumulation of mutations in vivo (Stalnacke et al, 1985).

When choosing a compound to analyze a molecule, it is important to choose wisely since many are bulky moieties that can influence cellular accumulation and activity (Zhang et al., 2001; Zhang et al., 2000). The research papers by Zhang et. al. 2000 and 2001 demonstrate that biodistribution patterns are heavily influenced by the labeling method. The different labels changed aspects of the behavior of an antisense molecule differently where a antisense molecule containing 99 mTc accumulated the quickest and a antisense molecule containing a cyclic anhydride of DTPA accumulated the slowest in cells. This is a good example demonstrating that modifications of the drug molecule can influence its overall availability and thus function. Bulky molecular groups have the ability to change the functionality of the molecule being studied by changing its inter- and intra-molecular interactions. The labeling groups attached to the nucleic acids can change the ability of these molecules to be recognized by cellular factors which could especially be a problem when the molecule must enter a natural cellular process. As described earlier, siRNA enter a pathway in the cell that is normally used for preventing transposon jumping, viral infection and regulating gene expression. As described above, the process of RNAi has many steps requiring a series of cellular factors to bind to the dsRNA and for the antisense strand of the dsRNA to bind its target and be recognized by cellular factors that induce cleavage of the mRNA. Attaching large fluorescent groups to a dsRNA could prevent it from reducing gene expression of the corresponding gene; therefore, it was unclear whether the functionality of dsRNA in RNAi would be compromised by labeling.

The analysis of labeled antisense molecules has shed light on their bioavalibility. Tracking unmodified antisense molecules has revealed the plasma half-life to be less than 5 minutes. Modifications to the antisense molecule have lead to a substantial increase in the bioavalibility. Labeling comes in handy to track the biodistribution pattern in vivo and in vitro. Following intravenous administration of radiolabeled antisense oligonucleotides, most of the molecule is found in liver and kidney. This biodistribution however can be changed by conjugating lipids, poly ethylene glycol, peptides, detergents and or antibodies (reviewed in Vyas et al., 2001 and applied antisense oligonucleotide technology 1998). All of these issues are very important when the molecules in question are potential drug candidates.

dsRNA have several benefits over currently used approaches to knock down gene expression. There is more of a chance for non-specific effects and less choices for target site selection with non-dsRNAs because effective antisense oligonucleotides often target elements that are critical for translation and mRNA processing, as well as regulatory elements (elements often shared by many messenger RNAs).

Functional dsRNA are relatively easy to identify and have longer lasting effects than antisense; also, compared to other nucleic acid molecules, they are more stable and require lower concentrations to induce gene silencing. Ribozymes are even more restricted to target sites in MRNA since the site of cleavage is determined by the ribozyme cleavage site specificity. RNAi technology also has advantages over recombination-based methods of knocking out genes in human cells. It is less time consuming and occurs at a much higher frequency within the population of cells in culture. Triplex forming oligonucleotides (TFO's), which are used to change the sequence of the chromosomal DNA, only work at efficiencies often in the maximum range of 5%. Successful use of TFO require the target cells to be dividing, restricting its use. Progress with TFO has been impeded by problems associated with target affinity, target accessibility, nuclease stability of the TFO, triplex stability and reliable assays for monitoring activity in vivo.

Thus, nucleic acids that are involved in RNAi are more desirable than other nucleic acids previously employed, such as antisense, ribozymes, and TFOs. Despite the research conducted using nucleotides that give rise to RNAi and despite the fact that nucleotides have been previously labeled, it was not known whether a nucleic acid molecule involved in RNAi could be labeled without eliminating its RNAi activity. A if labeled nucleic acid molecule could be used to effect RNAi and at the same time be used to evaluate its characteristics such as its localization, bioavailability, biodistribution, stability, and/or efficacy. Thus, there is a need for such labeled nucleic acids and ways of generating such labeled nucleic acids.

### SUMMARY OF THE INVENTION

The present invention is based on the inventors' discovery that a double stranded ribonucleic acid molecule capable of reducing the expression of a targeted gene through RNAi (referred to as “dsRNA” or “siRNA”) can be labeled yet maintain its RNAi activity. Thus, the present invention is directed to compositions and methods involving labeled dsRNA that effect, trigger, or induce RNAi. RNAi is mediated by an RNA-induced silencing complex (RISC), which associates (specifically binds one or more RISC components) with dsRNA of the invention and guides the dsRNA to its target mRNA through base-pairing interactions. Once the dsRNA is base-paired with its mRNA target, nucleases cleave the mRNA.

In some embodiments, the invention concerns a labeled dsRNA or siRNA that is capable of triggering RNA interference, a process by which a particular RNA sequence is destroyed. siRNA are dsRNA molecules that are 100 bases or fewer in length (or have 100 basepairs or fewer in its complementarity region). In some cases, it has a 2 nucleotide 3′ overhang and a 5′ phosphate. The particular RNA sequence is targeted as a result of the complementarity between the dsRNA and the particular RNA sequence. It will be understood that dsRNA or siRNA of the invention can effect at least a 20, 30, 40, 50, 60, 70, 80, 90 percent or more reduction of expression of a targeted RNA in a cell. dsRNA of the invention (the term “dsRNA” will be understood to include “siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. The complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous bases. In some embodiments, long dsRNA are employed in which “long” refers to dsRNA that are 1000 bases or longer (or 1000 basepairs or longer in complementarity region). The term “dsRNA” includes “long dsRNA” and “intermediate dsRNA” unless otherwise indicated. In some embodiments of the invention, dsRNA can exclude the use of siRNA, long dsRNA, and/or “intermediate” dsRNA (lengths of 100 to 1000 bases or basepairs in complementarity region).

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (see Sui et al., 2002 and Brummelkamp et al., 2002 in which a single strand with a hairpin loop is used as a dsRNA for RNAi). For convenience, lengths of dsRNA may be referred to in terms of bases, which simply refers to the length of a single strand or in terms of basepairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted MRNA is referred to as the “antisense strand” and the strand with a sequence identical to the targeted MRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

The single RNA strand or two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 31, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more (including the full-length of a particular's gene's MRNA without the poly-A tail) bases or basepairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or basepairs) or less, in which case they may also be referred to as “siRNA.” In specific embodiments the strand or strands of the dsRNA are less than 70 bases in length. With respect to those embodiments, the dsRNA strand or strands may be from 5-70, 10-65, 20-60, 30-55, 40-50 bases or basepairs in length. A dsRNA that has a complementarity region equal to or less than 30 basepairs (such as a single stranded hairpin RNA in which the stem or complementary portion is less than or equal to 30 basepairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 basepairs or fewer. In some cases, a dsRNA may be processed in the cell into siRNA.

The invention concerns labeled dsRNA. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C).

When two or more differentially colored labels are employed, fluorescent resonance energy transfer (FRET) techniques may be employed to characterize the dsRNA.

Labels contemplated for use in several embodiments are non-radioactive. In many embodiments of the invention, the labels are fluorescent, though they may be enzymatic, radioactive, or positron emitters. Fluorescent labels that may be used include, but are not limited to, BODIPY, Alexa Fluor, fluorescein, Oregon Green, tetramethylrhodamine, Texas Red, rhodamine, cyanine dye, or derivatives thereof The labels may also more specifically be Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, DAPI, 6-FAM, Fluorescein Isothiocyanate, HEX 6-JOE, Oregon Green 488, Oregon Green 500, Oregon Green 514, Pacific Blue, REG, Rhodamine Green, Rhodamine Red, Renographin, ROX, SYPRO, TAIA, TET, Tetramethylrhodamine, and/or Texas Red. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as Cy3, Cy5, or fluorescein (FAM). In still further embodiments, the labeling reagent is also incubated with a labeling buffer, which may be any buffer compatible with physiological function (i.e., buffers that is not toxic or harmful to a cell or cell component) (termed “physiological buffer”).

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

In addition to labeled, dsRNA compositions, the present invention further concerns methods for making such compositions. In some embodiments of the invention, methods for labeling a dsRNA or creating a dsRNA are included. These methods involve: hybridizing complementary sense and antisense RNA strands (or hybridizing a single strand with complementary regions) and labeling at least one strand or one portion of the strand. Any of the compositions described above may be implemented in methods of the invention. Thus, for example, the label may be fluorescent, the strands or the portions of a single strand may be differentially labeled, and/or the label may be at the 3′ and/or 5′ end, the label may be internal (that is, not at either end). In some embodiments, the method further includes first obtaining complementary RNA strands for RNAi or generating a single-stranded RNA with internal complementary regions.

In still further embodiments of the invention, the strand or strands are hybridized prior to labeling, while in other embodiments, labeling is done prior to hybridization. It is contemplated that methods of the invention may also include isolating or purifying the labeled or hybridized (or labeled and hybridized) RNA. In some cases, enzymes used for the labeling are later deactivated, such as through physical separation or heating.

A person of ordinary skill in the art is well aware of achieving hybridization of complementary regions or molecules. Such methods typically involve heat and slow cooling of temperature during incubation. As for labeling, such protocols are also well known. In some embodiments, maleimide-containing dyes suspended in DMSO are used to achieve a labeled RNA. These methods may also involve a buffer, such as ones that comprise one or more of the following: Hepes, Tris, phosphate, MOPS, or any other buffer appropriate for physiological applications, such as buffers with a pH of 7.0-7.5 and a concentration of 10-100 mM. In this pH range, thiol groups are sufficiently nucleophilic so that they react exclusively with the maleimide-containing dyes.

An end-labeled dsRNA may be achieved using an RNA with a 5′ thiophosphate moiety and incubating it with a thio-reactive fluorescent dye. Thio-reactive dyes include BODIPY, Alexa Fluor, fluorescein, HEX, Oregon Green, ROX, SYPRO, tetramethylrhodamine, Texas Red, cyanine dye, or derivatives thereof, which may be implemented in labeling protocols.

Labeling reactions may be incubated for at least or at most 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12 hours or more. Incubations may be at temperatures of 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90° C. or more, though it is specifically contemplated that temperatures may be in the range of 20° C. to 80° C., 25° C. to 75° C., or 60° C. to 70° C.

The present invention further concerns employing labeled dsRNA for RNA in a cell to evaluate biodistribution, transfection efficiency, targeting, localization, stability, or functionality of the labeled RNA strand. Methods for evaluating RNA interference in a cell are included as part of the invention. These methods include: a) introducing into the cell a fluorescently labeled, dsRNA molecule; and b) visualizing, identifying, or characterizing the fluorescently labeled, double-stranded RNA molecule. Any of the compositions described above may be implemented in these evaluative protocols. The dsRNA molecule may be introduced into the cell by methods known to those of ordinary skill in the art including various well known transfection protocols (lipid-mediated, calcium phosphate, electroporation, for example).

Furthermore, a person of ordinary skill in the art is well aware of ways of visualizing, identifying, and characterizing labeled nucleic acids, and accordingly, such protocols may be used as part of the invention. Examples of tools that may be used include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

Any cell that undergoes RNAi can be employed in methods of the invention. The cell may be a eukaryotic cell, mammalian cell such as a primate, rodent, rabbit, or human cell, a prokaryotic cell, or a plant cell. In some embodiments, the cell is alive, while in others the cell or cells is in an organism or tissue. Alternatively, the cell may be dead. The dead cell may also be fixed. In some cases, the cell is attached to a solid, non-reactive support such as a plate or petri dish. Such cells may be used for array analysis. It is contemplated that cells may be grown on an array and dsRNA administered to the cells.

The present invention also concerns kits for labeling and using dsRNA for RNA interference. Kits may comprise components, which may be individually packaged or placed in a container, such as a tube, bottle, vial, syringe, or other suitable container means. Kit embodiments include the one of more of the following components: labeling buffer comprising a physiological buffer with a pH range of 7.0 to 7.5; labeling reagent for labeling dsRNA with fluorescent label comprising an alkylating agent; control dsRNA comprising a dsRNA known to trigger RNAi in a cell, such as those disclosed herein, nuclease free water, ethanol, NaCl, reconstitution solution comprising DMSO or annealing buffer comprising Hepes and at least one salt. In further embodiments, the labeling reagent comprises Cy3, Cy5, and/or fluorescein (FAM).

The salt in the annealing buffer, in some embodiments, is potassium acetate and/or magnesium acetate. Annealing buffer may contain 1, 2, 3, 4, 5, 6, 7, 8, 9, 10, 15, 20, 25, 30, 35, 40, 45, 50, 100, 150, 200, 250, 300, 350, 400, 450, 500, 550, 600, 650, 700, 750, 800, 850, 900, 950, 1000, 2000, 3000, 4000, 5000 mM or more of a salt such as potassium acetate and/or magnesium acetate, and/or sodium acetate. It may also contain a buffer such as Hepes or Tris in a concentration of 10, 15, 20, 30, 40, 50, 60, 70, 80, 90, 100, 110, 120, 130, 140, 150, 160, 170, 180, 190, 200, 210, 220, 230, 240, 250 mM or more, with a pH in the range of 7.0-8.0. In one embodiment, a 5× concentration of annealing buffer comprises 150 mM Hepes, pH 7.4, 500 mM potassium acetate, and 10 mM magnesium acetate. Other concentrations may be adjusted accordingly. It is contemplated that kits may contain any component to create compositions of the invention and to implement methods of the invention.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Control dsRNA is included in some kit embodiments. Control dsRNA is dsRNA that can be used as a positive control for labeling and/or RNAi. The control may be provided as a single strand or as two strands.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A. Analysis of the ability of a siRNA against c-myc to knock down the expression of c-myc protein expression as compared to a scrambled siRNA or non-treated cells. FIG. 1B. The influence of a siRNA against c-myc to influence cell proliferation as compared to a scrambled siRNA and non-treated cells.

FIG. 2A. Analysis of c-myc protein expression by immunofluorescence of HeLa S3 cells either transfected with c-myc specific siRNA or a scrambled siRNA that contain one (single) or two (dual-labeled) of the strands of the siRNA duplex labeled at internal positions. FIG. 2B. HeLa S3 cells transfected with siRNA against c-myc with or without one of the two strands of the siRNA labeled or a scrambled siRNA that contains a label on one of the two strands of the siRNA were analyzed for defects in cell proliferation, which correlates with the relative ability of the siRNA against GAPDH to knock down c-myc expression. FIG. 2C. Analysis of HeLa S3 cells transfected with siRNA against GAPDH or a scrambled siRNA containing no label, one of the strands labeled or two of the strands labeled for defects in cell proliferation. Defects in cell proliferation also correlate with the ability of the siRNA against GAPDH to knock down GAPDH expression.

FIG. 3A. HeLa S3 cells transfected with siRNA against c-myc or GAPDH labeled at their 5′ ends were capable of knocking down the expression of their target genes as determined by immunofluorescence analysis. FIG. 3B. Non-labeled (same as “unlabeled”) or siRNAs labeled at one or both of their 5′ ends were transfected into HeLa S3 cells and cell proliferation rates were analyzed.

FIG. 4A. Non-labeled siRNA or siRNA labeled with a nucleic acid binding molecule DAPI were transfected into HeLa S3 cells and analyzed for the ability of the siRNA to influence cell proliferation rates. FIG. 4B. Non-labeled siRNA or siRNA labeled with a nucleic acid binding molecule DAPI were transfected into HeLa S3 cells and analyzed by immunofluorescence for the ability of the siRNAs to knock down gene expression. A “+” indicates low expression and “+++” indicates high expression.

FIG. 5A. Cy3-labeled dsRNA against Hrp48 (1.3 kb) and U2Af50 (1.2 kb) were added to Drosophila L2 cells at a final tissue culture concentration of 5 nM and analyzed using a fluorescent microscope. FIG. 5B. The ability of the long dsRNA to knock down the expression of their target mRNAs was analyzed using Northern blotting analysis.

FIG. 6A-B. siRNA labeled on the sense strand with FAM and Cy3 on the antisense strand were transfected into HeLa S3 cells. Forty-eight hours following transfection the siRNA was analyzed using a fluorescent microscope (FIG. 6A) or with a Leica confocal microscope (FIG. 6B). Arrows point to spots that are either entirely green or red indicating strand separation.

FIGS. 7A, B, C. siRNA labeled with Cy3 were transfected into HeLa S3 cells that were grown on cover slips. Forty-eight hours following transfection, the cells on the cover slips were fixed, mounted onto microscope slides and analyzed by fluorescent microscopy. The individual cells shown in FIGS. 7A, B and C are derived from a population of cells. The cells in FIGS. 7A and B appear to be in some state of division and the cell in FIG. 7C demonstrates the general localization of the siRNA in non-dividing cells.

FIG. 8. Analysis of transfection efficiency of GAPDH siRNA produced by chemical synthesis or enzymatic synthesis. The Cy3-labeled siRNAs were transfected into HeLa S3 cells on cover slips and twenty-four hours following transfection the cells were fixed and mounted onto glass slides and analyzed under the fluorescent microscope. Total cells in the field of view containing labeled siRNA were counted and divided by the total number of cells in that field to give the % of the cells transfected with or containing siRNA.

### DESCRIPTION OF ILLUSTRATIVE EMBODIMENTS

The present invention is directed to compositions and methods relating to a labeled nucleic acid molecule that can be used in the process of RNA interference (RNAi). RNAi results in a reduction of expression of a particular target. Double stranded RNA has been shown to reduce gene expression of a target. A portion of one strand of the double stranded RNA is complementary to a region of the target's MRNA while another portion of the double stranded RNA molecule is identical to the same region of the target's MRNA. As discussed earlier, the RNA molecule of the invention is double stranded, which may be accomplished through two separate strands or a single strand having one region complementary to another region of the same strand. Discussed below are uses for the present invention—compositions, methods, and kits—and ways of implementing the invention.

I. RNA Interference (RNAi)

RNA interference (also referred to as “RNA-mediated interference”)(RNAi) is a mechanism by which gene expression can be reduced or eliminated. Double stranded RNA (dsRNA) has been observed to mediate the reduction, which is a multi-step process. dsRNA activates post-transcriptional gene expression surveillance mechanisms that appear to function to defend cells from virus infection and transposon activity. (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene. (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals. (Grishok et al, 2000; Sharp, 1999; Sharp et al, 1999); Elbashir et al., 2001).

Interestingly, RNAi can be passed to progeny, both through injection into the gonad or by introduction into other parts of the body (including ingestion) followed by migration to the gonad. Several principles are worth noting (see Plasterk and Ketting, 2000). First, the dsRNA is typically directed to an exon, although some exceptions to this have been shown. Second, a homology threshold (probably about 80-85% over 200 bases) is required. Most tested sequences are 500 base pairs or greater, though sequences of 30 nucleotides or fewer evade the antiviral response in mammalian cells. (Baglioni et al., 1983; Williams, 1997). Third, the targeted mRNA is lost after RNAi. Fourth, the effect is non-stoichiometric, and thus incredibly potent. In fact, it has been estimated that only a few copies of dsRNA are required to knock down >95% of targeted gene expression in a cell. Fire et al. (1998).

Although the precise mechanism of RNAi is still unknown, the involvement of permanent gene modification or the disruption of transcription have been experimentally eliminated. It is now generally accepted that RNAᵢ acts post-transcriptionally, targeting RNA transcripts for degradation. It appears that both nuclear and cytoplasmic RNA can be targeted. (Bosher et al., 2000).

Some of the uses for RNAi include identifying genes that are essential for a particular biological pathway, identifying disease-causing genes, studying structure function relationships, and implementing therapeutics and diagnostics. As with other types of gene inhibitory compounds, such as antisense and triplex forming oligonucleotides, tracking these potential drugs in vivo and in vitro is important for drug development, pharmacokinetics, biodistribution, macro and microimaging metabolism and for gaining a basic understanding of how these compounds behave and function. siRNAs have high specificity and may perhaps be used to knock out the expression of a single allele of a dominantly mutated diseased gene.

A. Nucleic Acids for RNAi

The present invention concerns labeled, double-stranded RNA capable of triggering RNAi. The RNA may be synthesized chemically or it may be produced recombinantly. They may be subsequently isolated and/or purified.

As used herein, the term “dsRNA” refers to a double-stranded RNA molecule. The molecule may be a single strand with intra-strand complementarity such that two portions of the strand hybridize with each other or the molecule may be two separate RNA strands that are complementary to each other. It is contemplated that the region of complementarity in either case is at least 5 contiguous residues, though it is specifically contemplated that the region is at least or at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides. It is further understood that the length of complementarity between the dsRNA and the targeted mRNA may be any of the lengths identified above. Included within the term “dsRNA” is small interfering RNA (siRNA), which are generally 21-23 nucleotides in length.

dsRNA capable of triggering RNAi has one region that is complementary to the targeted mRNA sequence and another region that is identical to the targeted MRNA sequence. Of course, it is understood that an mRNA is derived from genomic sequences or a gene. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants.

A dsRNA may be of the following lengths, or be at least or at most of the following lengths: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It will be understood that these lengths refer either to a single strand of a two-stranded dsRNA molecule or to a single stranded dsRNA molecule having portions that form a double-stranded molecule.

The term “recombinant” may be used and this generally refers to a molecule that has been manipulated in vitro or that is the replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The use of “dsRNA” encompasses both “oligonucleotides” and “polynucleotides,” unless otherwise specified.

These definitions generally refer to a single-stranded molecule, but in specific embodiments will also encompass an additional strand that is partially (between 10 and 50% complementary across length of strand), substantially (greater than 50% but less than 100% complementary across length of strand) or fully complementary to the single-stranded molecule. Thus, dsRNA may encompass a molecule that comprises one or more complementary strand(s) or “complement(s)” of a particular sequence comprising a molecule.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequencers), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0. 15 M NaCl at temperatures of about 50° C. to about 70° C. It is understood that the temperature and ionic strength of a desired stringency are determined in part by the length of the particular nucleic acid(s), the length and nucleobase content of the target sequencers), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. In the table below, non-limiting, purine and pyrimidine derivatives and analogs are also provided.

TABLE 1
Purine and Pyrmidine Derivatives or Analogs

| Abbr. | Modified base description | Abbr. | Modified base description |
|---|---|---|---|
| ac4c | 4-acetylcytidine | Mam5s2u | 5-methoxyaminomethyl-2-thiouridine |
| Chm5u | 5-(carboxyhydroxylmethyl) uridine | Man q | Beta,D-mannosylqueosine |
| Cm | 2′-O-methylcytidine | Mcm5s2u | 5-methoxycarbonylmethyl-2-thiouridine |
| Cmnm5s2u | 5-carboxymethylamino-methyl-2-thioridine | Mcm5u | 5-methoxycarbonylmethyluridine |
| Cmnm5u | 5-carboxymethylaminomethyluridine | Mo5u | 5-methoxyuridine |
| D | Dihydrouridine | Ms2i6a | 2-methylthio-N6-isopentenyladenosine |
| Fm | 2′-O-methylpseudouridine | Ms2t6a | N-((9-beta-D-ribofuranosyl-2-methylthiopurine-6- |
| | | | yl)carbamoyl)threonine |
| Gal q | Beta,D-galactosylqueosine | Mt6a | N-((9-beta-D-ribofuranosylpurine-6-yl)N-methyl- |
| | | | carbamoyl)threonine |
| Gm | 2′-O-methylguanosine | Mv | Uridine-5-oxyacetic acid methylester |
| I | Inosine | o5u | Uridine-5-oxyacetic acid (v) |
| 16a | N6-isopentenyladenosine | Osyw | Wybutoxosine |
| mla | 1-methyladenosine | P | Pseudouridine |
| mlf | 1-methylpseudouridine | Q | Queosine |
| mlg | 1-methylguanosine | s2c | 2-thiocytidine |
| mlI | 1-methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | T | 5-methyluridine |
| m3c | 3-methylcytidine | t6a | N-((9-beta-D-ribofuranosylpurine-6- |
| | | | yl)carbamoyl)threonine |
| m5c | 5-methylcytidine | Tm | 2′-O-methyl-5-methyluridine |
| m6a | N6-methyladenosine | Um | 2′-O-methyluridine |
| m7g | 7-methylguanosine | Yw | Wybutosine |
| Mam5u | 5-methylaminomethyluridine | X | 3-(3-amino-3-carboxypropyl)uridine, (acp3)u |

A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Komberg and Baker, 1992).

3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. DsRNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moeity which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake and regulating RNA expression; U.S. Pat. No. 5,858,988, which describes hydrophobic carrier agent attached to the 2′-O position of oligonucleotides to enhanced their membrane permeability and stability; U.S. Pat. No. 5,214,136, which describes oligonucleotides conjugaged to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′deoxyguanosine nucleotides and nucleic acid analogs thereof).

B. Preparation of Nucleic Acids

A nucleic acid may be made by any technique known to one of ordinary skill in the art, such as for example, chemical synthesis, enzymatic production or biological production. Nucleic acid synthesis is performed according to standard methods. See, for example, Itakura and Riggs (1980). Additionally, U.S. Pat. No. 4,704,362, U.S. Pat. No. 5,221,619, and U.S. Pat. No. 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. Ser. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al 1989, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligonucleotides (Gillam et al., 1978; Gillam et al., 1979). Under controlled conditions, polynucleotide phosphorylase adds predominantly a single nucleotide to a short oligonucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic nucleic acid synthesizers.

Phosphoramidite chemistry (Beaucage and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Vector-based Technology. Alternatively, nucleic acids of the invention may be produced recombinantly. Recently, RNAi was effected using a DNA-vector based system in mammalian cells. (Sui et al., 2002; Brummelkamp et al., 2002). The resulting RNA is a single RNA molecule that has a region of complementarity such that the molecule forms a hairpin loop (and thus, qualifies as a dsRNA). Such nucleic acids may first be recombinantly produced and labeled in vitro or in vivo according to methods of the invention.

C. Nucleic Acid Transfer

Suitable methods for nucleic acid delivery to effect RNAi according to the present invention are believed to include virtually any method by which a nucleic acid (e.g., DNA, RNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon, carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

There are a number of ways in which expression vectors may be introduced into cells to generate dsRNA. In certain embodiments of the invention, the expression vector comprises a virus or engineered vector derived from a viral genome, while in other embodiments, it is a nonviral vector. Alternatively, other expression systems are also readily available.

II. Labels and Labeling Techniques

The present concerns double-stranded RNA involved in RNAi that is labeled. In many embodiments of the invention, the label is non-radioactive. A number of methods are known for labeling nucleic acids. Generally, nucleic acids may be labeled as single strands and then a strand or strands are incubated under conditions to allow complementary regions to hybridize to one another; alternatively, a double-stranded molecule may be labeled during or after hybridization.

A. Labeling Techniques

Examples of labeling include, but are not limited to, the following methods discussed below. For example, U.S. Pat. No. 6,262,252, which is specifically incorporated by reference discusses labeling nucleic acids with mustard or aziridine labeling reagents and may be used to label dsRNA. U.S. Pat. No. 5,728,525, which is specifically incorporated by reference, discusses some of these methods. Techniques for attaching labels have largely relied upon (a) functionalization of 5′ or 3′ termini of either the monomeric nucleosides or the oligonucleotide strands by numerous chemical reactions using deprotected oligonucleotides in aqueous or largely aqueous media (e.g., Cardullo et al., 1988); (b) synthesizing modified nucleosides containing (i) protected reactive groups, such as NH2, SH, CHO, or COOH, (ii) activatable monofunctional linkers, such as NHS esters, aldehydes, or hydrazides, or (iii) affinity binding groups, such as biotin, attached to either the heterocyclic base or the furanose moiety. Modifications have been made on intact oligonucleotides or to monomeric nucleosides which have subsequently been incorporated into oligonucleotides during chemical synthesis via terminal transferase or “nick translation” (see, e.g., Brumbaugh et al., 1988; Sproat et al., 1989; Allen et al., 1989); (c) use of suitably protected chemical moieties, which can be coupled at the 5′ terminus of protected olignnucleotides during chemical synthesis, e.g., 5′-aminohexyl-3′-O-phosphoramidite (Haralambidis et al., 1990); and, (d) addition of functional groups on the sugar moiety or in the phosphodiester backbone of the polymer (see Conway et al., 1989; Agrawal et al., 1990).

The simplest reactions have involved the attachment of non-nucleoside linkers and labels to the 3′ or 5′ end of existing oligonucleotides by either enzymatic or chemical methods. U.S. Pat. Nos. 6,376,179 and 5,573,913, both of which are incorporated by reference, specifically involve labeling of RNA with non-radioactive moieties. The former patent describes the use of terminal deoxynucleotidyl transferase (EC 2.7.7.31) to attach at least one 3′-terminal ribonucleotide to the 3′ end of a nucleic acid acceptor molecule. The latter patent discusses a number of other known methods for labeling RNA, such as the use of labeled nucleotides as a substrate for RNA polymerases (Langer, et al., 1981; Holtke et al., 1990). Nascent RNA can be labeled at its 5′ end using biotinylated dinucleotides as initiator oligonucleotides in an in vitro transcription reaction (Pitulle et al., 1992). In contrast, extant RNA molecules are end-labeled at their 5′ end through the use of polynucleotide kinase and [γ³²P]-ATP, i.e., radioactively (Richardson, 1981).

Sodja et al. (1978) describe the 3′-end-labeling of RNA molecules using biotin. By a multistep reaction, biotin is bound to the 3′-terminal ribose sugar of RNA via a NH₂(CH₅)NH₂ spacer or via cytochrome C. This reaction includes the oxidation of the sugar with periodate, reaction of the resulting dialdehyde with a NH₂ group of the spacer or of cytochrome C with formation of a Schiff base, a subsequent reduction with BH₄—, and covalent coupling of biotin to the spacer or to cytochrome C.

Poly(A) polymerase and T4 RNA ligase have been employed to produce a radioactive enzymatic 3′-end-labeled RNA. Poly(A) polymerase is used to attach [α³²p]-ATP to the 3′ end of RNA molecules (Winter et al., 1978). A [5′-³²P]-pCp can be attached to the 3′ ends of RNA molecules with T4 RNA ligase (Uhlenbeck et al., 1982). T4 RNA ligase can also be implemented to non-radioactively label RNA with biotin, tetramethylrhodamine and fluorescein derivatives of P¹-(6-aminohex-1-yl)-P²-(5′-adenosine)pyrophosphate (Richardson, et al., 1983).

In a publication by Roychoudhury and Kossel (1971), it is described that an oligodeoxynucleotide with two ribonucleotide units at the 3′ end can serve as a nucleic acid acceptor molecule for the attachment of dATP molecules by terminal deoxynucleotidyl transferase.

Terminal deoxynucleotidyl transferase (also denoted nucleoside triphosphate: DNA deoxynucleotidylexo-transferase or TdT) is an enzyme that occurs in the bone marrow and thymus of mammals and is obtainable for example from Boehringer Mannheim GmbH, Mannheim, Germany with a specific activity of ca. 60,000 U/mg.

U.S. Pat. No. 5,728,525 further discusses numerous methods for both cyclic and exocyclic derivatization of the N-nucleoside base, including the following:

Hapten labeling. DNA probes have been amino modified and subsequently derivatized to carry a hapten such as 2,4-dinitrophenol (DNP) to which enzyme-conjugated anti-hapten antibodies bind, which subsequently can be processed using a colorimetric substrate as a label (Keller et al., 1988).

Amino- and thiol-derivatized oligonucleotides. Takeda and Ikeda (1984) used phosphotriester derivatives of putresceinyl thyroidine to prepare amino-derived oligomers. Methods for synthesizing a deoxyuridine analog with a primary amine “linker arm” 12 carbons in length at C₅ have also been published (Jablonski et al., 1986). These were later reacted with fluorescein to produce a fluorescent molecule. U.S. Pat. No. 4,910,300) discusses pyrimidine derivatives on which the 6-amino group at C₄ is modified. 3′ and 5′ amino modifying phosphoramidites have been widely used in chemical synthesis or derivatized oligonucleotides and are commercially available.

Labeling with photobiotin and other biotinylating agents. The high affinity of biotin for avidin has been used to bind enzymatic or chemiluminescent reagents to derivatized DNA probes (Forster et al., 1985). Biotin conjugated to other linkers has also been widely used, including biotin-NHS esters (Bayer et al., 1980), biotin succinamides (Lee et al., 1984), and biotin maleimides (Bayer et al., 1985). Others have used biotin hydrazide to label the 4-amino group of cytidine (Reisfeld et al., 1987). U.S. Pat. No. 4,828,979) desribes such derivatizations at the 6-position of adenine, the 4-position of cytosine, and the 2-position of guanine. These derivatizations interfere with hydrogen bonding and base-pairing and have limited uses in producing oligomers for use in hybridization.

dU-Biotin labeling. Nucleoside 5′-triphosphates or 3′-O-phosphoramidites were modified with a biotin moiety conjugated to an aliphatic amino group at the 5-position of uracil (Langer et al., 1981; Saiki et al., 1985). The nucleotide triphosphate derivatives are effectively incorporated into double stranded DNA by standard techniques of “nick translation.” Once in an oligonucleotide, the residue may be bound by avidin, streptavidin, or anti-biotin antibody which can then be used for detection by fluorescence, chemiluminescence, or enzymatic processing.

11-digoxigenin-ddUTP labeling. The enzyme, terminal transferase, has been used to add a single digoxigenin-11-dideoxyUTP to the 3′ end of oligonucleotides. Following hybridization to target nucleic acids, DIG-ddUTP labeled hybridization probes were detected using anti-DIG antibody conjugate.

AAIF. Immunofluorescent detection can be done using monoclonal Fab′ fragments which are specific for RNA:DNA hybrids in which the probe has been derivatized with, e.g., biotin-11-UTP (Bobo et al., 1990; Viscidi et al., 1986).

Bisulfite modification of cytosine. Aliphatic amino groups have been introduced onto cytidine by a bisulfite catalyzed termination reaction; the amino groups were subsequently labeled with a fluorescent tag (Draper et al., 1980). In this procedure, the amino group is attached directly to the pyrimidine base. Like the derivatization of uracil, these derivatizations interfere with hydrogen bonding and base-pairing and are not necessarily useful for producing efficient hybridization oligomers.

Fluorophore derivatized DNA probes. Texas Red (Sulfochloro-Rhodamine) derivatized probes are commercially available which hybridize to specific target DNAs and which can be detected using a flow cytometer or a microscope. Numerous authors have reported coupling fluorophores to chemically synthesized oligonucleotides which carried a 5′ or 3′ terminal amino or thiol group (Brumbaugh et al., 1988).

Direct enzyme labeling. Chemical coupling of an enzyme directly to a chemically synthesized probe has been used for direct detection through substrate processing. For example, Urdea et al. described an oligonucleotide sandwich assay in which multiple DNA probe hybridizations were used to bind target DNA to a solid phase after which it was further labeled with additional, alkaline phosphatase-derivatized hybridization probes (Urdea et al., 1989).

Acridinium ester labeling. A single phenyl ester of methyl acridinium is attached at a central position on an RNA or DNA probe. Hydrolysis of the ester releases an acridone, CO₂, and light. Because the ester on unhybridized probes hydrolyzes more quickly than the ester on probes which have hybridized to target RNA or DNA, the chemiluminescence of the hybridized probes can be distinguished from that of free probes and is used in a “hybridization protection assay” (Weeks et al., 1983).

Methods for derivatization of the furanose ring and at the phosphodiester backbone of oligonucleotides have been reported.

Internucleotide linkage reporter groups (R₁₀ site). Phosphorothioate esters have been used to provide a binding site for fluorophores such as monobromobimane (Conway et al., 1989). Agrawal and Zamecnik (1990) reported methods for incorporating amine specific reporter groups (e.g., monobromobimane) and thiol specific reporter groups (e.g., fluorescein isothiocyanate) through modifying the phosphodiester backbone of DNA to phosphoramidites and phosphorothioate diesters, respectively.

Glycosidic reporter groups (R₁₁ through R₁₄ sites). U.S. Pat. No. 4,849,513 describes the syntheses for an assortment of derivatives and labels on the glycosidic moiety of nucleosides and nucleoside analogs through the introduction of an aliphatic amino group at R₁₀. The authors did not report or claim any uses or applications of inherently fluorescent oligonucleotides, either made chemically or enzymatically or using the fluorescent nucleoside analogs or their derivatives.

B. Fluorescent Labels

The fluorescent labels contemplated for use as conjugates include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX 6-JOE, Oregon Green 488, Oregon Green 500, Oregon Green 514, Pacific Blue, REG, Rhodamine Green, Rhodamine Red, Renographin, ROX, SYPRO, TAMA, TET, Tetramethylrhodamine, and/or Texas Red.

It is contemplated that dsRNA may be labeled with two different labels. Furthermore, fluorescence resonance energy transfer (FRET) may be employed in methods of the invention ( e.g., Klostermeier et al., 2002; Emptage, 2001; Didenko, 2001, each incorporated by reference).

C. Visualization Techniques

A number of techniques for visualizing or detecting labeled dsRNA are readily available. The reference by Stanley T. Crooke, 2000 has a discussion of such techniques (Chapter 6) which is incorporated by reference. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997, spectroscopy, capillary gel electrophoresis (Cummins et al., 1996), spectroscopy; mass spectroscopy; radiological techniques; and mass balance techniques.

III. Kits

Any of the compositions described herein may be comprised in a kit. In a non-limiting example, reagents for labeling a dsRNA are included in a kit. The kit may further include reagents for creating or synthesizing the dsRNA. The kits will thus comprise, in suitable container means, an enzyme for labeling the dsRNA and/or the label. It may also include one or more buffers, such as labeling buffer or a hybridization buffer, compounds for preparing the dsRNA, and components for isolating the resultant labeled dsRNA. Other kits of the invention may include components for making a nucleic acid array comprising dsRNA, and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, erg., injection and/or blow-molded plastic container s into which the desired vials are retained.

Such kits may also include components that facilitate isolation of the labeled dsRNA. It may also include components that preserve or maintain the dsRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following in addition to labeling reagents, which may include a reactive dye (such as an alkylating agent attached to the dye):

1) Control dsRNA, including but not limited to, GAPDH siRNA or c-myc siRNA (shown in Examples);

2) Nuclease-free water;

3) RNase-free containers, such as 1.5 ml tubes;

4) RNase-free elution tubes;

5) glycogen;

6) ethanol;

7) sodium acetate;

8) ammonium acetate;

9) agarose;

10) nucleic acid size marker;

11) RNase-free tube tips;

12) and RNase or DNase inhibitors.

It is contemplated that such reagents are embodiments of kits of the invention. Such kits, however, are not limited to the particular items identified above and may include any labeling reagent or reagent that promotes or facilitates the labeling of a nucleic acid to trigger RNAi.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### dsRNA Effects Reduction in c-myc Expression, which Leads to a Decrease in Cell Proliferation

HeLa cells were transfected with a chemically synthesized siRNA made against the 3′UTR of c-myc (sense: 5′-CGAUUCCUUCUAACAGAAAdTdT-3′ (SEQ ID NO:1) and anti-sense: 5′-UUUCUGUUAGAA GGAAUCGdTdT-3′ (SEQ ID NO:2)) or a chemically synthesized scrambled siRNA (sense: 5′-GCGACGUU CCUGAAACCACdTdT-3′(SEQ ID NO:3) and scrambled antisense: 5′-GUGGUUUCAGGAACGUCGCdTdT-3′(SEQ ID NO:4)). To hybridize the RNAs, 1.5 nanomoles of each of the sense and anti-sense RNAs were mixed in a solution comprising 100 mM KOAc, 30 mM HEPES-KOH pH 7.4, and 2 mM MgOAc. The solution was incubated at 95° C. for one minute and then at 37° C. for one hour.

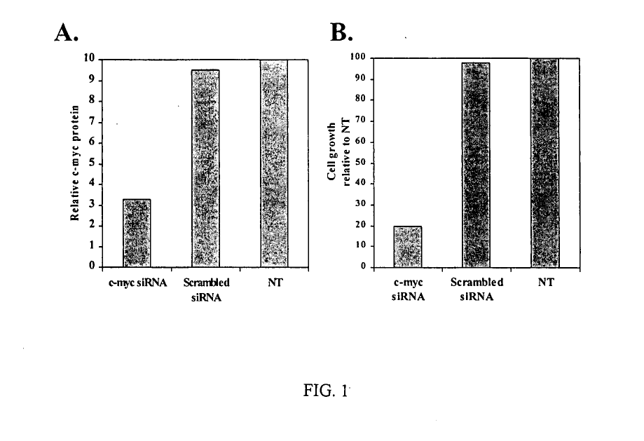

To confirm that the majority of the RNA was double-stranded, a fraction of the hybridized RNAs was evaluated on a 12% acrylamide gel. The transfections were done as follows: 5×10⁴HeLa S3 cells were plated on a 24-well dish in DMEM supplemented with 10% FBS. The cells were then incubated overnight at 37° C. in a humidified 5% CO₂ incubator. The synthetic siRNA stock was then diluted into 40 μl OptiMEM (Invitrogen) to give a 100 nM final in 250 μl total volume per well. For each well, 1.5 μl of Oligofectamine (Invitrogen) was added to OptiMEM to give a final volume of 7.5 μl. The diluted oligofectamine was added to the diluted siRNA and incubated at room temperature for 15 minutes. Then, medium was aspirated, 200 μl fresh growth medium added to each well, approximately 50 μl of oligonucleotide complexes was overlaid onto cells and incubated at 37° C. in a humidified 5% CO₂ incubator overnight. Cell proliferation was determined using AlamarBlue agent (Biosource international, Inc. CA catalog # DAL1025) (FIG. 1A). To perform the AlamarBlue assay, AlamarBlue reagent was added into the tissue culture media at 10% final concentration. The mixture was incubated for 3-6 hours in growth conditions after which fluorescence was quantified using the spectra Max GeminiXS (molecular Devices, Sunnyvale, CA) (FIG. 1B).

Protein expression of c-myc was determined using Immunofluorescence. For immunofluorescence, HeLa cells plated in 12-well dishes were grown on cover slips in DMEM/10% FBS and transfected as described above. Forty-eight hours after transfection the cells were fixed with 4% paraformaldehyde/PBS, permeabilized by exposure to 0.1% Triton X-100/PBS for 5 min and incubated with 3% BSA in PBS for 1 hr. The cells were incubated with a mouse anti-myc monoclonal antibody (Neomarkers cat# 67P05) at a 1:200 dilution in PBS for 1 hour and were washed briefly with PBS. The cells were then incubated with fluorescein-conjugated goat anti-mouse IgG (Jackson ImmunoResearch Laboratories cat # 715-095-150) for 1 hour. The cells were mounted with Vectashield™ containing DAPI (Vector Laboratories catalog number H-1200) and the images were analyzed using an Olympus BX60 microscope and acquired and analyzed with the Hitachi KP-c571 camera and Adobe® Photoshopg and quantified using Scion Image. This data clearly demonstrates that c-myc siRNA specifically reduces c-myc protein expression and causes cell growth defects. Cell proliferation defects are consistent with previous reports in which c-myc is down-regulated using antisense molecules (Kimura et al., 1995).

### Example 2

### Fluorescent Internally-Labeled siRNA Reduces the Expression of Target Genes in Mammalian Cells as Effectively as Unlabeled siRNA

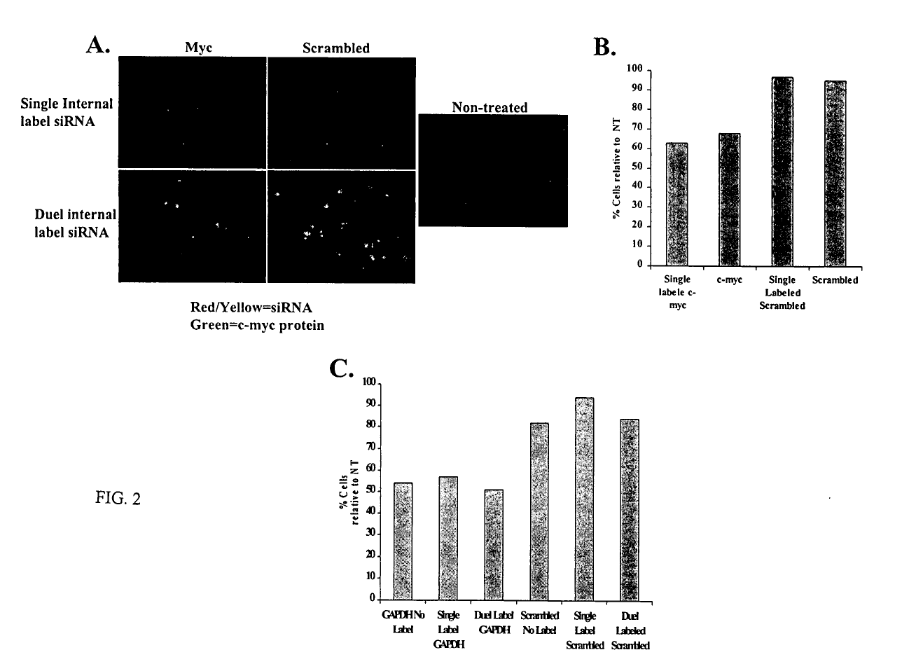

The ability of labeled siRNA to function at inducing RNAi in mammalian cells was determined by analyzing protein expression and cell proliferation defects. HeLa cells were transfected with a chemically synthesized siRNA made against the 3′UTR of c-myc (sense: 5′-CGAUUCCUUCUAACAGAAAdTdT-3′ (SEQ ID NO:1) and anti-sense: 5′-UUUCUGUUAGAAGGAAUCGdTdT-3′ (SEQ ID NO:2)) a c-myc scrambled siRNA (sense: 5′-GCGACGUUCCUGAAACCACdTdT-3′ (SEQ ID NO:3) and chemically synthesized scrambled antisense: 5′-GUGGUUUCAGGAACGUCGCdTdT-3′ (SEQ ID NO:4)) or GAPDH (Gap sense: 5′-GGUCAUCCAUGACAACUUUdTdT-3′(SEQ ID NO:5) and Gap anti-sense: 5′-AAAGUU GUCAUGGAUGACCdTdT-3′ (SEQ ID NO:6)) and a chemically synthesized GAPDH scrambled siRNA (sense 5′-ACUACCGUUGUUAUA GGUGdTdT-3′ (SEQ ID NO:7) and antisense 3′-dTdTUGAUGGCAACAAUAUCCAC-5′ (SEQ ID NO:8)) for FIG. 2C. The siRNA to c-myc and a scrambled control siRNA were labeled at internal locations using the Rhodamine, Fluorescein (FIGS. 2A and 2B) and Fluorescein and cy3 (FIG. 2C). To generate the labeled Rhodamine siRNA the LabelIT kit produced by Mirus corp (cat # MIR 3925) was used. Fluorescein- and cy3-labeled siRNA were made as follows: in a microcentrifuge tube 5 μl of labeling buffer (200 mM MOPS pH7.5), 14.5 μl of 50 μM 21 mer single strand RNA oligonucleotide stock (5 μg total), 7.5 μl of cy3 (Mirus cat #MIR 3600) and Fluorescein (Mirus cat #MIR 3200) LabelIt reagent and 23 μl of nuclease free water were added. The reaction mix was incubated at 37° C. for 1 hour, ethanol precipitated and suspend in 14.5 μl of nuclease free water. Following labeling of the single stranded siRNA, it was hybridized to its complement, which was either labeled—giving the duel labeled siRNA—or was unlabeled—giving an siRNA labeled on a single strand. The siRNA were transfected using oligofectamine Gibo-BRL (cat #12252011) at a concentration of 100 nM final siRNA concentration in the tissue culture media as described in Example 1. After 48 hours, the cells were analyzed using immunofluorescence staining using the method described in FIG. 1A in Example 1 or by analyzing cell number (FIG. 1B). Cell number was determined by manual counting of trypsinized cells with a hemocytometer. All transfections were done at least in duplicate and reductions in cell numbers are statistically significant and the variation in cell number observed between siRNAs containing no label one label or two labels is not significant. FIG. 2C shows the comparison of the abilities of GAPDH siRNA containing a single label, double label or no label to induce RNAi in mammalian cells. The siRNAs containing internal fluorescent labels were as functional as siRNAs containing no labels. To rule out the possibility that the unlabeled siRNA was what was causing the effect, the efficiency of labeling was analyzed using RNA band shift analysis, where labeled RNA migrates slower than unlabeled siRNA. Using this method, approximately 50% of the siRNA are labeled. If labeled siRNA were not functional then we would expect a 50% reduction in the amount of effect that the labeled siRNAs have decreasing c-myc expression.

### Example 3

### 5′-End Fluorescent-Labeled siRNA Knocks Down the Expression of the Gene for which it Corresponds as Effectively as Unlabeled siRNA

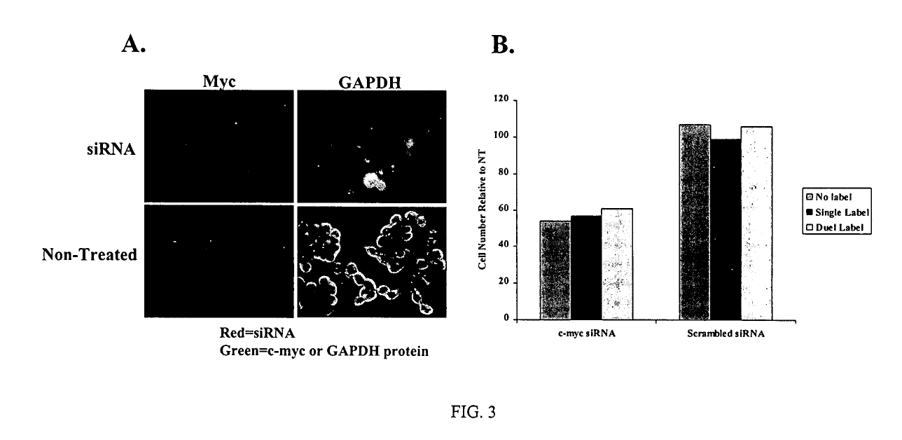

The functionality of siRNA containing 5′ labeled ends was then tested in mammalian cells (FIGS. 3A-B). Using a 5′ end Labeling Kit (Vector Laboratories cat #MB-9001) a 5′-end labeled siRNA was chemically synthesized corresponding to both c-myc (same sequence as that listed in Example 1) and GAPDH (Gap sense: 5′-GGUCAUCCAUGACAACUUUdTdT-3′ (SEQ ID NO:5) and Gap anti-sense: 5′-AAAGUUGUCAUGGUGACCdTdT-3′ (SEQ ID NO:6)) with either fluorescein and/or Texas red. To label the siRNAs at the 5′end, a thiophosphate was transferred from ATPγS to the 5′ hydroxyl group of the sense or anti-sense strand of the siRNA using T4 polynucleotide kinase. The gamma phosphate containing the reactive sulfur group was added by combining 2 μl of universal reaction buffer, 12 μl of 50 [μM stock of siRNA, 1 μl of ATPγS, 2 μl of T4 polynucleotide kinase in a micro-centrifuge tube, 3 μl of nuclease free water and reactions are incubated for 30 minutes at 37° C. Following the addition of the gamma phosphate of ATPγS, the dye was attached by adding 10 μl of the thio-reactive either fluorescein or Texas red. The reaction was mixed, incubated for 30 minutes at 65° C., Phenol extracted, ethanol precipitated and suspended in 12 μl of water. In cases where the siRNAs are dual labeled, the sense and anti-sense strands both containing fluorescent labels were hybridized as described in Example 1. Where siRNAs contain a single label, one labeled siRNA was hybridized to a complementary unlabeled siRNA using the procedure described in Example 1. Each dye was tested on each strand of the siRNA and no difference in functionality of the siRNA was observed; data for Texas Red are shown in FIG. 3. siRNAs described above were labeled with Texas Red, transfected into HeLa cells, and analyzed by immunofluorescence (FIG. 3A). c-myc siRNA labeled on one or on each 5′ end or unlabeled siRNA were transfected into HeLa S3 cells and effects on proliferation were analyzed (FIG. 3B). It did not make a difference which strand contained the specific dye.

### Example 4

### SiRNA Labeled in its Minor Groove Knock Down Expression of the Gene for Which it Corresponds as Efficiently as siRNA that is Unlabeled

DAPI (4′6′-Diamidino-2-phenylindole Dihydrochloride: Hydrate), a minor groove nucleic acid binding molecule, was used to label chemically synthesized c-myc siRNA (sense: 5′-CGAUUCCUUCUAACAGAAAdTdT-3′ (SEQ ID NO:1) and anti-sense: 5′-UUUCUGUUAGAAGGAAUCGdTdT-3′ (SEQ ID NO:2)) and scrambled (sense: 5′-GCGACGWUCCUGAAACCACdTdT-3′ (SEQ ID NO:3) and scrambled antisense: 5′-GUGGUUUCAGGAACGUCGCdTdT-3′ (SEQ ID NO:4)) siRNAs. To label the siRNAs with DAPI, DAPI was incubated with the c-myc and a scrambled siRNA to yield a final concentration of 0.1 mg/ml of DAPI in nuclease free-water for 15 minutes at 37° C. The sample was then precipitated using ethanol and suspended in nuclease-free water to bring the final siRNA concentration to 20 mM.

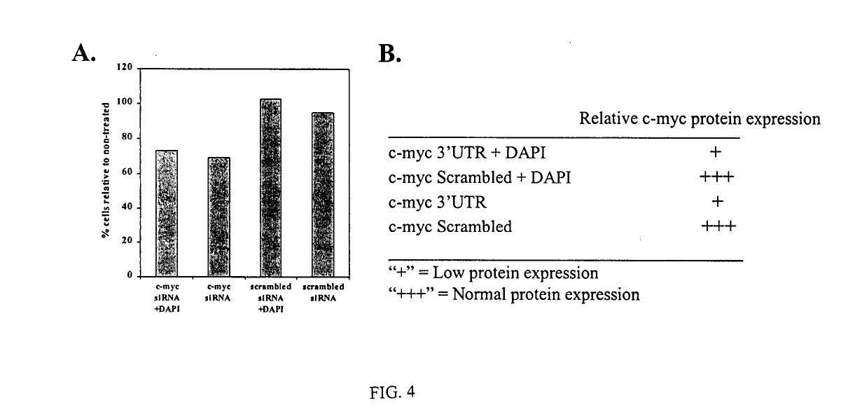

Following DAPI staining, these siRNAs were transfected as follows. First, HeLa S3 cells at 5×10³ cells/well were plated in DMEM/10% FBS and incubated overnight at 37° C. in a humidified 5% CO₂ incubator. The siRNA stock was then diluted into 40 μl OptiMEM (Invitrogen) to give a 100 nM final concentration in 250 μl total volume per well. Next, 1.5 μl of Oligofectamine (Invitrogen) was mixed with OptiMEM for a final volume of 7.5 μl and mixed with the diluted siRNA. The sample was then incubated at room temperature for 15 minutes following which the mixture was added to cells containing 250 μl of DMEM/10% FBS. Forty-eight hours following transfection, the samples were analyzed for cell proliferation (FIG. 4A) as described in Example 2 and for protein expression using immunofluorescence (FIG. 4B) as described in Example 1. The siRNA labeled with DAPI are as effective at reducing the expression of its target gene as is the unlabeled siRNA. The DAPI labeling efficiency was determined to be 100% using band shift analysis. This rules out any possibility that the functionality of the labeled siRNA was due to the unlabeled siRNA in the population.

### Example 5

### Labeled dsRNA Greater than 1 kb Knocks Down Expression as Efficiently as Unlabeled dsRNA

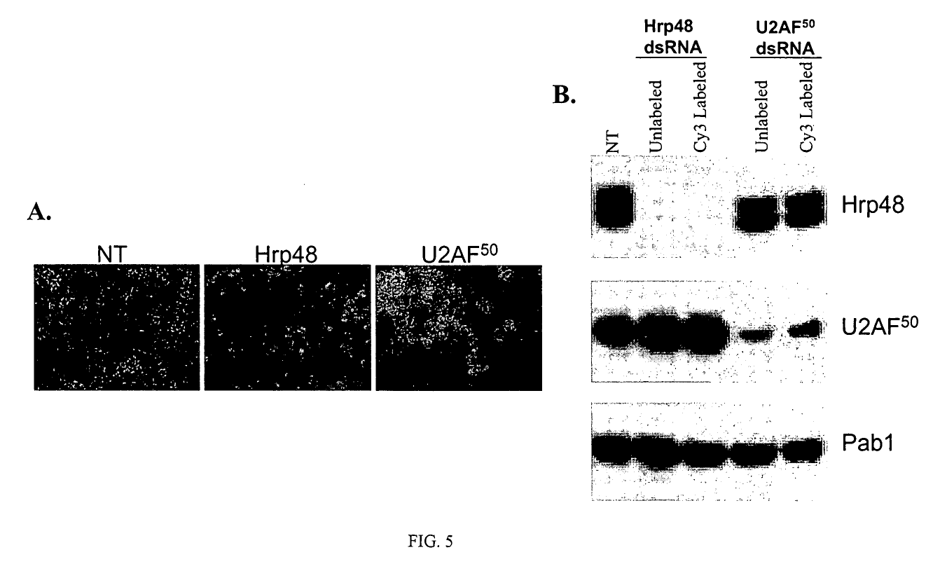

Labeled dsRNAs greater than 1000 bases (as opposed to siRNA) are capable of inducing RNAi in Drosophila L2 cells. FIG. 5A. Long dsRNAs corresponding to the genes for Hrp⁴⁸ (1.3 kb dsRNA) (GenBank Accession No. X62639, which is incorporated by reference) and U2AF50 (1.2 kb dsRNA) (GenBank Accession No. NG000299, which is incorporated by reference) were labeled with Cy3 using the LabeiIT kit produced by Mirus Corp. (cat # M[R 3925) using 5 μl of the Label IT reagent. The resulting products were added to 5×10⁴ L2 cells attached to cover slips in SF-M media (Gibco cat # 10797-025) at a final concentration of 10 nM. Forty-eight hours following the addition of the dsRNA, the cells were harvested and the labeled long dsRNA was analyzed using Olympus BX60 microscope and acquired and analyzed with the Hitachi KP-c571 camera and Adobe® Photoshop®. FIG. 5B. Long dsRNAs corresponding to the genes for Hrp48 and U2AF50 labeled with Cy3 using the LabelIT kit produced by Mirus Corp. (cat # MIR 3925) or unlabeled long dsRNA corresponding to these same genes were added to 5×10⁴ L2 cells in SF-M media (Gibco cat # 10797-025) at a final concentration of 10 nM. Forty-eight hours following transfection, the cells were harvested by trypsinization and lysed in 50 mM HEPES pH 8.3, 430 mM KCl, 0.1% NP-40 and 1 mM EDTA. Total protein was determined using a Bradford assay and equal amounts of protein were loaded on to a denaturing acrylamide gel containing SDS. Protein was detected using Western blotting procedure with an antibody to the indicated proteins.

### Example 6

### Labeled siRNA can be used to Elucidate Mechanism of RNAi

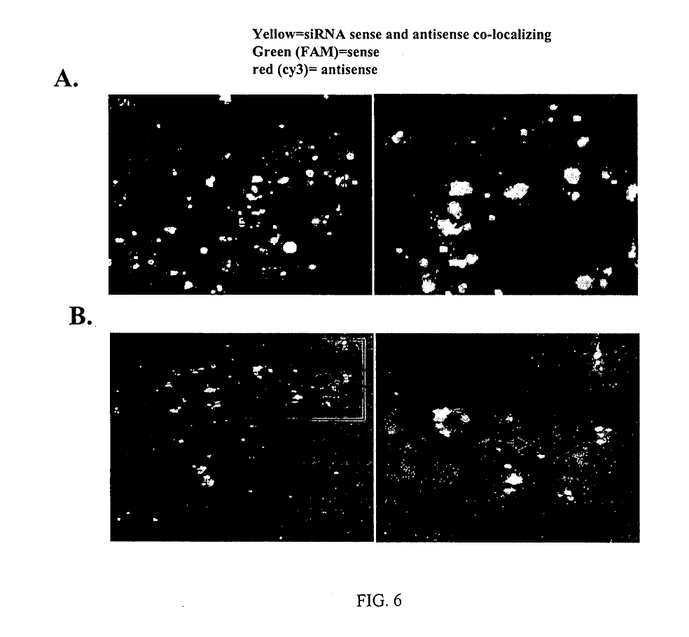

Labeled siRNA was used to analyze whether the sense and antisense strands of siRNA separate in vivo as has been described in vitro (Nykanen et al., 2001). An siRNA against the 3′UTR of c-myc was labeled according to the procedure described in Example 2 where the sense strand of the siRNA was labeled with FAM and the antisense strand of the siRNA was labeled with Cy3. The dual labeled siRNA was transfected into HeLa S3 cells using oligofectamine Gibo-BRL (cat #12252011) according to their recommendations at 100 nM final siRNA concentration in the tissue culture media. Forty-eight hours following transfection, the siRNA was analyzed using Olympus BX60 microscope in FIG. 6A or using confocal microscope analysis in FIG. 6B. A clear separation between the sense and antisense strands of the siRNA was observed, as many of the spots that were detected were solely green or red. Arrows point to individual red and green spots. When a dual labeled siRNA is double stranded it can be seen as a yellow where the sense and antisense strands separate they are observed as red or green. The strand separation observed here was confirmed using Fluorescence Resonance Energy Transfer (FRET) analysis.

### Example 7

### Using Labeled siRNA to Analyze Cellular siRNA Distribution

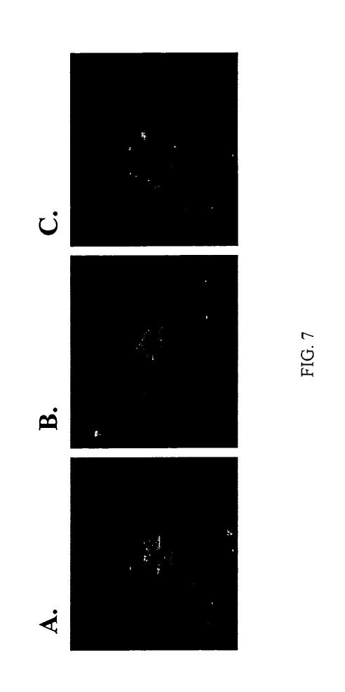

The analysis of siRNA distribution revealed that siRNA are localized near the nuclear periphery and that the siRNA appear to be passed to daughter cells after division. The distribution of siRNA in a dividing HeLa S3 in FIGS. 7A and 7B and in HeLa in FIG. 7C. The cells were grown on cover slips in a 24-well tissue culture plate and were transfected with Cy3 labeled GAPDG siRNA with the sequence described in Example 1 in FIGS. 1A and 1B or with a cy3 labeled β-actin siRNA in HeLa cells using oligofectamine. Forty-eight hours following transfection the samples were fixed using 4% paraformaldehyde/PBS, mounted onto slides with Vectashield™ containing DAPI (Vector Laboratories catalog number H-1200), and analyzed using the Olympus BX60 microscope. Dividing cells that contained siRNA were detected in a population of cells on cover slips by observing chromosome condensation and nuclear separation/reformation. In all cases where the cell appeared to have just undergone a division event, the siRNA was localized to the central region of the dividing cell where it is separate from the DAPI stained DNA.

### Example 8

### Transfection Efficiency using cy3-Labeled GAPDH siRNA

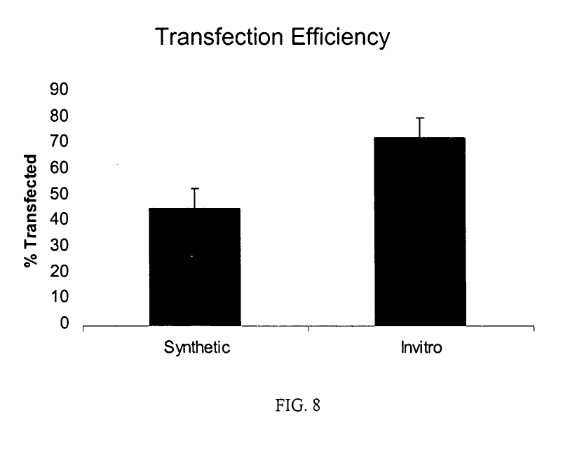

Shown as FIG. 8 is a graph that compares the transfection efficiency of the same GAPDH siRNA made by either chemical synthesis or using Ambion's Silencer siRNA construction kit (cat# 1620). Enzymatically synthesized siRNA were produced from DNA templates (Gap 484 sense) 5′-AAAA AGTTGTCATGGATGACCCCTGTCTC -3′ (SEQ ID NO:7) and (GAP 484 antisense) 5′-AAGGTCATCCATGACAACTTTCCTGTCTC-3′ (SEQ ID NO:8) where these oligonucleotides were hybridized with a T7 promoter primer that is supplied in the Silencer siRNA construction kit in hybridization buffer (20 mM Tris, pH 7.0, 100 mM NaCl, 1 mM EDTA, pH 8.0; pH to 7.0) at 70° C. for 5 minutes and then left at room temperature for 5 minutes. Following hybridization, the hybridized oligonucleotides were filled in using Klenow DNA polymerase in the presence of dNTP (5 mM dATP, 5mM dCTP, 5 mM dGTP, 5 mM dTTP in 10 mM Tris, pH 8.0) and Klenow 10× reaction buffer (500 mM Tris pH 7.0, 100 mM MgCl₂, 50 mM DTT; pH to 7.0) supplied in Ambion's Silencer siRNA kit. The siRNA were then transcribed from the filled in DNA templates where 2 μl of each filled in siRNA template was mixed with 10 μl of a 2× NTP mix (15 mM ATP, 15 mM CTP, 15 mM GTP, 10 mM alpha-thio UTP in 10 mM Tris pH 7.5.), 2 μl of 10× T7 reaction buffer (400 mM Tris pH 8.0, 240-260 mM MgCl₂, 20 mM Spermidine, 100 mM DTT), 4 μl water, and 2 μl of enzyme mix (T7 RNA polymerase 200 U/μl, IPP (inorganic pyrophosphatase) 0.05U/μl, RNase Inhibitor 0.3 U/μl, Superase In® 2U/μl from Ambion #2694, 1% CHAPS), all of which are supplied in the kit. The reaction was incubated at 37° C. for 2 hours and the sense and antisense reactions were combined and further incubated overnight. The transcription reaction was then treated with 2.5 μl Dnase (10 U/μl) and 3 μl RNase SA (15,000U/μl ) with 6 μl of digestion buffer (100 mM Tris, pH 7.5, 25 mM MgCl₂, 5 mM CaCl₂), and 48.5 μl of water at 37° C. for 1 hour. 400 μl of binding buffer (1.6 M NaCl solution added to 4 ml ethanol) was then added to the nuclease digestion and incubated at room temperature for 5 minutes and added to a filter cartridge pre wet with 100 μl of siRNA wash buffer (1 M NaCl solution added to 9 ml ethanol. The digested siRNA transcription reaction was then added to the column, washed two times with 500 μl of siRNA wash buffer and eluted in 100 μl of nuclease free-water and quantified. Gap484 siRNA was transfected into 24-well plates containing HeLa S3 cells (final concentration of about 100 nM siRNA final concentration) on cover slips using oligofectamine a final concentration of 100 nM. Forty-eight hours following transfection the samples were fixed using 4% paraformaldyhyde/PBS, mounted onto slides using with Vectashield™ containing DAPI (Vector Laboratories catalog number H-1200) and analyzed using the Olympus BX60 microscope. The number of cells containing siRNAs compared to the number of cells lacking siRNA was divided and graphed.

### Example 9

### Labeled siRNA Produced by Chemical Synthesis will be Compared to Unlabeled, Chemically Synthesized siRNA

Labeled siRNA that are made by chemical synthesis will be tested for activity as compared to siRNA that are unlabeled or labeled using post-synthesis techniques as described in Example 2, 3, and 4. To perform this study, tests will be conducted using siRNAs against c-myc, GAPDH and their scrambled controls that have labels at their 5′ or 3′ ends or at internal locations that are added using an oligonucleotide synthesizer. These labeled siRNAs will be tested for activity as compared to that of siRNAs labeled at 5, 3′ ends and at internal locations added using post synthesis methods. This will demonstrate that siRNAs that are labeled using chemical synthesis methods can also be found as active as unlabeled siRNAs or siRNAs labeled using post labeling techniques.

### Example 10

### Attachment of other Bulky Groups to siRNA will be Tested for Increased or Decreased siRNA Activity

Further tests will be conducted to determine whether attaching modifications such as lipids or cholesterol, PEG, or additional groups will influence the activity of siRNA. The siRNAs will be chemically modified during chemical synthesis. These siRNAs will then be tested for activity relative to an unmodified siRNA by transfecting the molecules into cells with or without a transfection agent. The modified siRNAs will be analyzed for activity under the same circumstances as positive and negative control siRNA that do not contain labels.

### Example 11

### siRNA Stability, Distribution and Function in Mice using Fluorescent Labeled siRNA

siRNA will be labeled with fluorescent molecules using the silencer siRNA labeling kit cy3 and Fluorescein. The kit, in some embodiments, will contain the following components: 10× Labeling Buffer (200 mM MOPS pH 7.5); Reconstitution Solution (DMSO); Nuclease-free Water; 5 M NaCl; 5× siRNA Annealing Buffer (150 mM Hepes, pH 7.4, 500 mM potassium acetate, 10 mM magnesium acetate); control siRNA (GAPDH siRNA in 20 μM solution, which is approximately 10 μg total); Cy3 Labeling Reagent (40 μg of dried Cy3 powder) and/or Fluorescein Labeling Reagent (40 μg of dried fluorescein powder).

Fluorescein and cy3 labeled siRNA will be made as follows: in a microcentrifuge tube 5 μl of 10× labeling buffer (200 mM MOPS pH 7.5), 19.2 μl of 20 μM 21 mer double stranded RNA oligonucleotide stock (5 μg total), 7.5 μl of cy3 (Mirus cat #MIR 3600) and Fluorescein (Mirus cat #MIR 3200) LabelIt reagent and nuclease free water will be added. The reaction mix will be incubated at 37° C. for 1 hour, ethanol precipitated and suspended in 19.2 μl of nuclease free water. The labeled siRNA will then be injected into animals. At intervals following the injection, the organs from the animals will be analyzed using fluorescent microscopy for labeled siRNA. This will provide information on both the times in which siRNA can be detected in the organism, to what organs the siRNA is distributed, and whether the fluorescent siRNA is functional in vivo.

### Example 12

### Ambion Instructions Manual

The following constitutes excerpts from Ambion Inc.'s Instructions Manual that is included with a kit for labeling dsRNA of the invention. The excerpts are included by way of example of protocols for using compositions and methods of the invention. It should be understand that siRNA includes any dsRNA for RNAi and that dsRNA may be a single stranded siRNA or comprise two separate complementary strands.

I. siRNA (any dsRNA for RNAi) Labeling Protocol

A. Labeling reaction

1. Add 100 μl of Reconstitution Solution to Labeling Reagent and mix well. Using RNase-free barrier tips, add 100 μl of Reconstitution Solution to the dry Cy3 or Fluorescein (FAM) Labeling Reagent. To ensure that the Labeling Reagent is fully suspended, vortex the tube after adding Reconstitution Solution, then let the mixture sit at room temperature for 5 min and vortex again. Store reconstituted Labeling Reagent at −20° C.

2. Assemble the labeling reaction and mix well. The Labeling Reaction may be scaled up or down depending on the amount of nucleic acid to be labeled. However, the Labeling Reagent should never constitute more than 20% of the total reaction volume. The time and temperature of incubation, and the amount of Labeling Reagent all influence labeling efficiency (or specific activity). In a sterile, nuclease-free tube, assemble the reagents in the listed order making sure to add the Labeling Reagent last. Mix well by vortexing. Limit exposure of the reaction mixture to light for the entire procedure.

a. Duplex siRNA (5 μg)

| Amount | Component |
|---|---|
| 18.3 μl | Nuclease-free Water |
| 5.0 μl | 10 × Labeling Buffer |
| 19.2 μl | 21 mer duplex siRNA at 20 μm (˜5 μg) |
| 7.5 μl | Cy3 or FAM Labeling Reagent |

b. Single-stranded siRNA (5 μg)

| Amount | Component |
|---|---|
| 22.5 μl | Nuclease-free Water |
| 5.0 μl | 10 × Labeling Buffer |
| 15 μl | 21 mer ssRNA oligonucleotide at 50 μm (˜5 μg) |
| 7.5 μl | Cy3 or FAM Labeling Reagent |

3. Incubate at 37° C. for 1 hr. Incubate the reaction mix at a constant temperature of 37° C. for 1 hr.

B. Ethanol Precipitation of Labeled RNA

Labeled RNA should be kept away from light as much as possible. Since the siRNA will be used in tissue culture, use aseptic technique while handling it. Autoclave any tubes used to hold the siRNA.

The inventors do not observe short term toxicity from transfection of siRNA that has not been ethanol precipitated, however, genetic alterations may occur if cells are exposed to unreacted Labeling Reagent for long periods of time. This ethanol precipitation removes unreacted Labeling Reagent, and the inventors recommend including it.

1 . Add 0.1 vol 5 M NaCl, and 2.5 vol 100% ethanol. For the 50 μl reactions described in section II.A. Labeling reaction on page 4, add the following and mix well:

5 μl 5MNaCl (0.1 volume)

125 μl cold 100% ethanol (2.5 volumes)

2. Place at −20° C. for 30-60 min. After mixing well, place the mixture at −20° C. (or colder) for 30-60 min. The labeled RNA precipitates during this incubation.

3. Centrifuge at top speed 10 min, discard supernatant.

a. Pellet the labeled RNA by high speed centrifugation for 10-20 min.

Use the top speed compatible with your tubes; this centrifugation should be ≧8,000 ×g.

b. Carefully remove the supernatant; avoid disrupting the pellet.

c. A red (Cy3) or green (FAM) siRNA pellet should be visible.

4. Wash pellet with 175 μl 70% ethanol.

a. Gently add 175 μl 70% ethanol making sure not to disrupt the pellet, and centrifuge at ≧8,000 ×g for 5 min (use the top speed compatible with your tubes).

b. Carefully remove all traces of supernatant with a pipette.

5. Dry at room temp 5-10 min. Dry the RNA at room temperature for 5-10 min. Do not dry the pellet for longer than 5 [symbol] 10 min or it will be difficult to solubilize.

6. Suspend the RNA pellet in Nuclease-free Water or buffer. Resuspend the RNA pellet in Nuclease-free Water or in the buffer of your choice. If desired, resuspend the RNA to its volume before the labeling reaction (e.g. 19.2 μl for duplex siRNA or 15 μl for ssRNA oligonucleotide) to finish with the same nucleic acid concentration. Labeled ssRNA that will be hybridized to make double-stranded siRNA should be suspended in Nuclease-free Water.

Since the labeled siRNA will be used in tissue culture, filter sterilize buffer before using it to resuspend the labeled siRNA. A small amount of RNA may be lost during the ethanol precipitation, so if your application requires extremely accurate quantitation of the labeled siRNA, measure the RNA concentration by spectrophotometry.

II. Troubleshooting

A. Positive Control Reaction

The GAPDH siRNA is a duplex siRNA supplied at 20 [μM. It is provided so that users can verify that the kit is working properly.

Also, the GAPDH siRNA has been transfected using Ambion's Silencer Transfection Reagents to knock down the expression of GAPDH in several common cell lines.

1. Positive control labeling reaction.

a. Label 19.2 μl of the GAPDH siRNA following the instructions for duplex siRNA in section I.A.2.a above.

b. Continue the procedure through the ethanol precipitation in section I.B above.

Resuspend the labeled GAPDH siRNA in 19.2 μl Nuclease-free Water.

2. Analysis of the positive control labeling reaction. Check the labeling of the GAPDH siRNA by spectrophotometry, or by electrophoresis.

At least 25% of the GAPDH siRNA should be labeled; this corresponds to a base:dye ratio of 84.

a. Analysis of the positive control labeling reaction by spectrophotometry

i. Dilute the labeled GAPDH siRNA solution with 76.8 μl 200 mM MOPS pH 7.5 (adjust the pH with NaOH). This is a 1:5 dilution.

ii. Measure the absorbance of the labeled RNA at 260 nm and at the absorbance maximum for the fluorescent dye (550 nm for Cy3, or 492 nm for FAM). As a baseline, also record the A260 and the A550 or 492 of the 200 mM MOPS used to dilute the labeled siRNA.

iii. Calculate the base:dye ratio using the calculator on the web site at [http:l/www.ambion.com/techlib/append/base_dye. html], or do the calculation as described in section V.E below.

b. Analysis of the positive control labeling reaction by electrophoresis (See section V.D below for recipes and gel running instructions.)

i. Put 19.2 μl of unlabeled GAPDH siRNA (supplied with the kit) in a nuclease-free tube, add gel loading buffer, and mix well. This sample will be used for comparison with the labeled GAPDH siRNA.

ii. Add gel loading buffer to the 19.2 μl of labeled GAPDH siRNA from step 1.b, and mix well.

iii. Load both samples on a 20% acrylamide gel, and run the gel until the bromophenol blue (the faster migrating dye) has migrated about ¾ of the way through the gel.

iv. Visualize the siRNA by ethidium bromide staining; labeled siRNA will migrate slower in the gel than unlableled siRNA.

Acrylamide gel analysis of the Silencer siRNA Labeling positive control experiment. The GAPDH siRNA supplied with the kit was labeled with FAM, and run on a 20% acrylamide gel. This is the reverse image of the ethidium bromide stained gel. Labeled siRNA runs slower than unlabeled siRNA. Using gel documentation system software to compare the band intensity of labeled and unlabeled GAPDH siRNA reveals that about 45% of the RNA was labeled in this experiment. A fraction of the labeled siRNA contains more than one dye molecule; this produces a pale ladder of bands migrating more slowly than the band representing siRNA labeled with a single dye molecule.

B. No Colored Pellet is Visible after Ethanol Precipitation

Normally, the siRNA can be visualized at the bottom of the tube after ethanol precipitation (step I.B.3) as a red (Cy3) or green (FAM) pellet. If a colored pellet is not visible, but the GAPDH siRNA supplied with the kit labels as expected, consider the following troubleshooting suggestions.

1. The siRNA is degraded. Check the integrity of the labeled siRNA by running a 2.5 μg sample of the RNA on a 20% acrylamide gel. Also consider running an equal amount of unlabeled siRNA in an adjacent lane. The labeled siRNA should migrate slower than unlabeled siRNA, but should appear intact.

2. There is too little RNA to see. Make sure that enough RNA was included in the reaction by checking the A₂₆₀ of the input RNA solution. Also be very careful when removing the supernatant after the ethanol precipitation to avoid dislodging the pellet.

3. The precipitation did not work well. Be sure to follow the recommended conditions, and incubation/centrifugation times, in section I.B. Ethanol Precipitation of Labeled RNA for optimal precipitation.

C. The siRNA Cannot Be Detected in Transfected Cells

1. Transfection of the labeled siRNA was poor. If transfection efficiency is too low, the labeled siRNA will not be detectable in transfected cells. There are many parameters that can affect transfection efficiency, check the literature and any documentation provided with your transfection agent for transfection troubleshooting and optimization suggestions.

2. The siRNA was not adequately labeled. Check whether the siRNA was labeled either by running it on 20% acrylamide gel (section III.D), or using a spectrophotometer (section III.E).

If labeling efficiency is below 25% the labeled siRNA could be difficult to detect in transfected cells by fluorescent microscopy.

D. Cell Cultures Become Contaminated After Transfection

Since the siRNA will be used in tissue culture, use aseptic technique while handling it. Autoclave any tubes that used to hold the siRNA, and if you use a solution other than the Nuclease-free Water supplied with the kit to resuspend the labeled siRNA, filter sterilize it.

If cell cultures appear to be contaminated as a result of transfection with the labeled siRNA, test each individual component in the kit in your tissue culture system. Also test whether your siRNA alone introduces contamination.

If necessary, the labeled siRNA can be filter sterilized just before preparing transfection agent:siRNA complexes for transfection.

III. Additional Procedures

A. Annealing RNA Oligonucleotides to Make siRNA

1. Mix equal amounts of each RNA oligonucleotide in 1× Annealing Buffer.

a. In an RNase-free tube combine 50 μM solutions of the sense and antisense RNA oligonucleotides and 5× siRNA Annealing Buffer for a final concentration of 20 μM each RNA strand and 1× siRNA Annealing Buffer.

Example: To make 75 μl of a 20 μM duplex siRNA:

| Amount | Component |
|---|---|
| 30 μl | Sense RNA oligonucleotide |
| 30 μl | Antisense RNA oligonucleotide |
| 15 μl | 5 × siRNA Annealing Buffer |
| 75 μl | total |

b. Vortex to mix, then spin briefly to collect the contents at the bottom of the tube.

2. Incubate 1 min at 90° C. Heat the mixture to 90° C. for 1 min/50-100 μl solution in a preheated heat block to denature any secondary structure in the RNA oligonucleotides. If the volume of the siRNA annealing mixture is >100 μl increase the incubation time at 90° C. proportionally (e.g a 150 μl mixture should be incubated for ˜2 min).

3. Incubate 1 hr at 37° C. The 1 hr 37° C. incubation allows the RNA oligonucleotides to anneal slowly so that they form a perfect duplex siRNA.

4. Store at −20° C. Once annealed, duplex siRNA is much more nuclease resistant than ssRNA and can be safely stored frozen at −20° C. in a non-frost free freezer for 6 months or longer.

B. Suspension of Dry RNA Oligonucleotides

Treat gloves and surrounding area with an RNase decontamination agent such as RNaseZap® prior to starting the work. Use RNase-free tubes and tips for all manipulations.

Oligonucleotides are often supplied dry; briefly centrifuge tubes to ensure that the dried oligonucleotide is at the bottom of the tube. The specification sheet provided by oligonucleotide manufacturers often contains the following information:

moles synthesized

mass amount synthesized

OD₂₆₀ units synthesized

The following sets of calculations explain how to make a 50 μM solution of an RNA oligonucleotide for use in the Silencer siRNA Labeling procedure.

These calculations can also be done automagically using the calculator on the web site. Find it at http://www.ambion.com/techlib/append/oligo_dilution.html.

Once the ssRNA is in solution, store it at −20° C. for up to a few months, or at −80° C. for extended periods of time.

TABLE 2
RNA Oligonucleotide Conversions

| | |
|---|---|
| Average MW* of ssRNA1 | # of nt × 320 |
| Average MW of dsRNA | # of nt × 640 |
| MW of 21 mer ssRNA | 6.7 μg/nmole |
| MW of 21 mer dsRNA | 13.4 μg/nmole |
| 50 μM solution of 21 mer ssRNA | 0.33 μg/μl |
| 20 μM solution of 21 mer dsRNA | 0.26 μg/μl |
| 1 A₂₆₀ of 21 mer ssRNA | 33 μg/ml |
| 50 μM | 0.05 nmoles/μl |

*MW = molecular weight

1. Making a 50 μM oligonucleotide solution based on the nmoles synthesized. Calculate the amount of Nuclease-free Water to add as follows:

$$\begin{matrix}\frac{nmoles synthesized}{0.05 nmoles \text{/} μl} = μ l for suspension to give a \\ 50 μ M solution \\ Example : \\ \frac{25 nmoles synthesized}{0.05 nmoles / μl} = 500 μ l for suspension to give \\ a 50 μ M solution\end{matrix}$$

2. Making a 50 μM oligonucleotide solution based on the mass amount of RNA synthesized. If the specification sheet for the oligonucleotide provides only the mass amount of RNA oligonucleotide synthesized, this is how to calculate the suspension volume necessary to make a 50 μM solution.

a. Determine the molecular weight of the RNA oligonucleotide:

$$\begin{matrix}Mw = 320 g \text{/} mol per base \times 21 bases = 6720 g \text{/} mole \\ = 6.7 μg \text{/} nmole\end{matrix}$$

b. Calculate the molar amount of RNA synthesized. The molar amount of RNA synthesized is the mass amount divided by the molecular weight.

$$\# nmoles synthesized = \frac{μ g synthesized}{6.7 μg \text{/} nmole}$$

c. Calculate the amount of Nuclease-free Water for suspension as in section B.1 on page 12

$$\begin{matrix}\frac{nmoles synthesized}{0.05 nmoles / μl} = μl for suspension to give a \\ 50 μM solution \\ d . Example : & \\ \frac{167.5 μg synthesized}{6.7 μg \text{/} nmole} = 25 nmoles synthesized & \\ \frac{25 nmoles}{0.05 nmoles \text{/} μl} = 500 μl for suspension to give a \\ 50 μM solution & \end{matrix}$$

C. Calculating the Volume of ssRNA or dsRNA Needed for the Procedure

1. Calculating the volume of a molar solution of duplex siRNA needed for 5 jig. The Silencer siRNA Labeling procedure uses 5 μg of siRNA. The following calculation shows how to determine what volume of a duplex siRNA solution of known molarity contains 5 μg of RNA.

a. Calculate the concentration of the RNA oligonucleotide solution

concentration=(MW)×(molarity of the solution)

b. Divide the mass amount desired by the concentration of the solution

c. Example: 100 μM solution:

$$\begin{matrix}MW = 320 g \text{/} mol per base \times 21 bases = 6720 g \text{/} mole \\ = 6720 μg \text{/} μmole\end{matrix}$$

100 μM solution=1—μmoles/L

Concentration=6720 μg/μmole×100 μmole=6.72×10⁶ μg/L

$$\frac{5 μg}{6.72 \times {10}^{5} μg \text{/} L} = 7.44 \times {10}^{- 6} L = 7.44 μl of a 100 μM$$

$$solution is 5 μg of RNA$$

2. Calculating the volume of a molar solution of RNA oligonucleotide needed for 5 μg. The Silencer siRNA Labeling procedure uses 5 μg of RNA. The following calculation shows how to determine what volume of a single-strand RNA oligonucleotide solution of known molarity contains 5 μg of RNA.

a. Calculate the concentration of the RNA oligonucleotide solution

concentration=(MW)×(molarity of the solution)

b. Divide the mass amount desired by the concentration of the solution

c. Example: 100 μM solution

$$\begin{matrix}MW = 640 g \text{/} mol per base \times 21 bases = 13440 g \text{/} mole \\ = 1.344 \times {10}^{4} μg \text{/} μmole\end{matrix}$$

100 μM solution=100 μmoles/L

concentration=1.344×10⁴ μg/μmole×μmoles/L=1.344×10⁶ μg/L

$$\frac{5 μg}{1.344 \times {10}^{6} μg \text{/} L} = 7.44 \times {10}^{- 6} L = 7.44 μl of a 100 μM solution$$

$$is 5 μg of RNA \\ = 3.72 \times {10}^{- 6} L = 3.72 μl of a 100 μM solution$$

$$is 5 μg of RNA$$

D. Acrylamide Gel Electrophoresis

1. 6× non-denaturing gel loading buffer

| Concentration | Component | For 10 ml | |
|---|---|---|---|
| 37% | Glycerol (100%) | 3.7 | ml |
| 0.025% | Bromophenyl blue | 2.5 | mg |
| 0.025% | Xylene cyanol | 2.5 | mg |
| 20 mM | 1 M Tris-HCl, pH 8 | 100 | μl |
| 5 mM | 500 mM EDTA | 100 | μl |
| | Nuclease-free water | To 10 | ml |

Alternatively, Ambion offers an all-purpose Gel Loading Solution for native gels, Cat #8556; this 10× solution is rigorously tested for nuclease contamination and functionality.

2. 10× TBE. TBE is generally used at 1× final concentration for preparing gels and/or for gel running buffer.

| Concentration | Component | For 1 L | |
|---|---|---|---|
| 0.9 M | Trisbase | 109 | g |
| 0.9 M | Boric Acid | 55 | g |
| 20 mM | 0.5 M EDTA solution | 40 | ml |

Dissolve with stirring in about 850 ml nuclease-free water. Adjust the final volume to 1 L.

Alternatively, Ambion offers 10× TBE as a ready-to-resuspend mixture of ultrapure molecular biology grade reagents (Ambion Cat #9863). Each packet makes 1 L of 10× TBE.

3. 20% Non-denaturing Acrylamide Gel Mix. The inventors suggest running gels that are approximately 15 cm long to adequately resolve the labeled and unlabeled RNA. 15 ml is enough gel solution for one 13×15 cm×0.75 mm gel

| For 15 ml | | Component |
|---|---|---|
| 1.5 | ml | 10X TBE |
| 7.5 | ml | 40% acrylamide (acryl: bis-acryl = 19:1) |
| | | (c.g. Ambion Cat #9022) |
| to 15 | ml | High quality water |

Stir at room temperature to thoroughly mix, then add:

| | |
|---|---|
| 120 μl | 10% ammonium persulfate |
| 16 μl | TEMED |

Mix briefly after adding the last 2 ingredients, which will catalyze polymerization, then pour gel immediately. (It is not necessary to treat with diethylpyrocarbonate)

4. Gel set up and sample loading.

Follow the manufacturers instructions for the details of attaching gels to the running apparatus.

Use 1× TBE as the gel running buffer.

Add non-denaturing gel loading buffer to the samples to 1×, mix well and load the samples in the gel wells.

5. Electrophoresis conditions. Run gels at ˜250 Volts constant voltage until the bromophenol blue (the faster-migrating dye) has moved about ¾ of the length of the gel.

6. Stain the gel in 0.5-1 μg/ml ethidium bromide in water or 1× TBE. Stain the nucleic acids in the gel by soaking for 5-10 min in 0.5-1 μg/ml ethidium bromide in water or 1× TBE. Visualize the gel on a UV transilluminator.

E. Measuring Base:Dye Ratio and RNA Concentration by Spectrophotometry

TABLE 3
Reference values for spectrophotometry calculations

| | Absorbance | Extinction | | |
|---|---|---|---|---|
| | maxium | coefficient | Dye correction factor | MW_{base} |
| Cy3 | 550 | 150,000 | 0.08 | 1296 |
| FAM | 492 | 68,000 | 0.32 | 1006 |
| 21-mer | 260 | 9,700 | — | 320 |
| single | | | | |
| stranded | | | | |
| dsRNA_{base} | | | | |
| 21-mer two- | 260 | 8,000 | — | 320 |
| stranded | | | | |
| dsRNA_{base} | | | | |

1. Measuring the base:dye ratio.

a. Dilute the labeled RNA 5 [symbol] 10 fold in 200 mM MOPS pH 7.5 (adjust the pH with NaOH).

b. Blank the spectrophotometer with the 200 mM MOPS at 260 nm, and at the maximum absorbance wavelength for the dye (A_{dye}): 550 nm for Cy3 or 492 nm for FAM.

c. Measure the absorbance of the diluted RNA 260 nm, and at the maximum absorbance wavelength for the dye (A_{dye}) 550 nm for Cy3 or 492 nm for FAM. With these absorbance values in hand, use the convenient base:dye ratio calculator on the web site to do the math. Find it at:

http://www.ambion.com/techlib/append/base_dye. html.

d. Since Cy3 and FAM absorb some light at 260 nm (as well as at their absorbance maxima), remove their contribution to the A₂₆₀ reading with the following calculation and the appropriate dye correction factor from Table 2

A_{base}=(A₂₆₀×dilution factor)−(A_{dye}×dilution factor×dye correction factor)

e. Calculate the ratio of bases to dye molecules using the following equation:

$$Base dye = \frac{( {A}_{base} \times extinction {coefficient}_{dye} )}{( {A}_{dye} \times extinction {coefficient}_{base} )}$$

2. Calculating the concentration of the nucleic acid: Using values from Table 2 and the corrected absorbance of the labeled RNA from step 1.d above, the concentration of the labeled RNA can be calculated using the following equation:

$$Mg \text{/} ml RNA = \frac{( {A}_{base} \times {MW}_{base} )}{( extinction {coefficient}_{base} \times path length in cm}$$

Most spectrophotometers have a 1 cm path length; if you don't know the path length for the spectrophotometer, assume that it is 1 cm.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.

U.S. Pat. No. 4,659,774

U.S. Pat. No. 4,682,195

U.S. Pat. No. 4,683,202

U.S. Pat. No. 4,684,611

U.S. Pat. No. 4,704,362

U.S. Pat. No. 4,816,571

U.S. Pat. No. 4,828,979

U.S. Pat. No. 4,849,513

U.S. Pat. No. 4,910,300

U.S. Pat. No. 4,952,500

U.S. Pat. No. 4,959,463

U.S. Pat. No. 5,141,813

U.S. Pat. No. 5,214,136

U.S. Pat. No. 5,221,619

U.S. Pat. No. 5,223,618

U.S. Pat. No. 5,264,566

U.S. Pat. No. 5,302,523

U.S. Pat. No. 5,322,783

U.S. Pat. No. 5,378,825

U.S. Pat. No. 5,384,253

U.S. Pat. No. 5,428,148

U.S. Pat. No. 5,446,137

U.S. Pat. No. 5,464,765

U.S. Pat. No. 5,466,786

U.S. Pat. No. 5,470,967

U.S. Pat. No. 5,480,980

U.S. Pat. No. 5,538,877

U.S. Pat. No. 5,538,880

U.S. Pat. No. 5,550,318

U.S. Pat. No. 5,554,744

U.S. Pat. No. 5,563,055

U.S. Pat. No. 5,563,055

U.S. Pat. No. 5,573,913

U.S. Pat. No. 5,574,146

U.S. Pat. No. 5,580,859

U.S. Pat. No. 5,583,013

U.S. Pat. No. 5,589,466

U.S. Pat. No. 5,591,616

U.S. Pat. No. 5,602,240

U.S. Pat. No. 5,602,244

U.S. Pat. No. 5,610,042

U.S. Pat. No. 5,610,289

U.S. Pat. No. 5,614,617

U.S. Pat. No. 5,623,070

U.S. Pat. No. 5,637,683

U.S. Pat. No. 5,645,897

U.S. Pat. No. 5,652,099

U.S. Pat. No. 5,656,610

U.S. Pat. No. 5,670,663

U.S. Pat. No. 5,672,697

U.S. Pat. No. 5,681,947

U.S. Pat. No. 5,700,922

U.S. Pat. No. 5,702,932

U.S. Pat. No. 5,705,629

U.S. Pat. No. 5,708,154

U.S. Pat. No. 5,714,606

U.S. Pat. No. 5,728,525

U.S. Pat. No. 5,736,524

U.S. Pat. No. 5,763,167

U.S. Pat. No. 5,777,092

U.S. Pat. No. 5,780,448

U.S. Pat. No. 5,789,215

U.S. Pat. No. 5,792,847

U.S. Pat. No. 5,858,988

U.S. Pat. No. 5,859,221

U.S. Pat. No. 5,872,232

U.S. Pat. No. 5,886,165

U.S. Pat. No. 5,945,100

U.S. Pat. No. 5,981,274

U.S. Pat. No. 5,994,624

U.S. Pat. No. 6,251,666

U.S. Pat. No. 6,262,252

U.S. Pat. No. 6,376,179

PCT Application No. WO 94/09699

PCT Application No. WO 95/06128

European Appl. 266,032

Allen et al., Biochemistry, 28:4601-4607, 1989.

Antisense Drug Technology Stanley T. Crooke (ed) Marcel Dekker and Co, Basel, Switzerland, Chapter 6, 2001.

Agrawal and Zamecnik, Nucleic Acids Research, 18(18):5419-5423, 1990.

Baglioni and Nilson, Interferon, 5:23-42, 1983.

Bayer and Wilchek, Methods of Biochemical Analysis, 26:1-45, 1980.

Bayer et al, Analytical Biochemistry, 149:529-536, 1985.

Beaucage, and Lyer, Tetrahedron, 48:2223-2311, 1992.

Bernstein et al., Nature, 409: 363-366, 2001.

Bobo et al., In: Diagnosis of Chlamydia trachomatis Cervical Infection by Detection of Amplified DNA with an Enzyme Immunoassay, 1990.

Bosher and Labouesse, Nat. Cell Biol., 2:E3 1-E36, 2000.

Brumbaugh et al. Proc Natl Acad Sci USA, 85(15):5610-5614, 1988.

Brummelkamp et al., Science, 296(5567):550-553, 2002

Caplen et al., Proc Natl Acad Sci USA, 98: 9742-9747, 2001.

Cardullo et al., Proc Natl Acad Sci USA, 85(23):8790-8794, 1988

Chen and Okayama, Mol. Cell Biol., 7(8):2745-2752, 1987.

Cogoni. and Macino, Natur,e 399:166-169, 1999.

Cogoni, C., and Macino, Science, 286:342-2344, 1999.

Conway et al., Nucleic Acids Res. Symposium Series, 21:43-44, 1989.

Cummins et al., In IRT: Nucleosides and nucleosides, La Jolla CA, pp 72, 1996.

Dalmay eta., Cell, 101:543-553,2000.

Dalmay et al. EMBO J. 20:2069-2078, 2001.

Didenko, Biotechniques, 31(5): 1106-16, 1118, 1120-1, 2001.

Dewanjee et al., Biotechniques, 5: 844-846, 1994.

Draper and Gold, Biochemistry, 19:1774-1781, 1980.

Elbashir et al, Nature, 411:494-498, 2001.

Emptage et al.,: Neuron, 2001 Jan;29(1):197-208, 2001.

Fechheimer et al., Proc. Nat'l Acad. Sci. USA, 84:8463-8467, 1987.

Fire et al., Nature, 391:806-811, 1998.

Fraley et al., Proc. Nat'l Acad Sci. USA, 76:3348-3352, 1979.

Forster et al. Nucleic Acids Research, 13(3):745-761, 1985.

Froehler et al., Nucleic Acids Res. 14(13):5399-5407, 1986.

Gillam et al., J. Biol. Chem., 253:2532, 1978.

Gillam et al., Nucleic Acids Res., 6:2973, 1979.

Gopal, Mol. Cell Biol., 5:1188-1190, 1985.

Graham and Van Der Eb, Virology, 52:456-467, 1973.

Griffey et al., J Mass Spectrom, 32(3):305-13, 1997.

Grishok et al., Cell, 106: 23-34, 2001.

Hamilton and Baulcombe, Science, 286:950-952, 1999.

Hammond et al., Nat Rev Genet, 2(2): 110-9, 2001.

Haralambidis et al., Nucleic Acids Res, 18(3):493-9, 1990.

Harland and Weintraub, J. Cell Biol., 101:1094-1099, 1985.

Holtke and Kessler, Nucleic Acids Res, 18(19):5843-51, 1990.

Hutvagner et al., Science, 293:834-838, 2001.

Itakura and Riggs, Science, 209:1401-1405, 1980.

Itakura et al., J. Biol. Chem., 250:4592 1975.

Jablonski et al., Nucleic Acids Research, 14(15):6115-6128, 1986.

Kaeppler et al., Plant Cell Reports 9: 415-418, 1990.

Kaneda et al., Science, 243:375-378, 1989.

Kato et al, J. Biol. Chem., 266:3361-3364, 1991.

Keller et al., Analytical Biochemistry, 170:441-450, 1988.

Kettinget al., Cell, 99:133-141, 1999.

Khorana, Science, 203, 614 1979.

Kimura et al., Cancer Research, 55:1379-1384, 1995.

Klostermeier and Millar, Biopolymers, 61(3): 159-79, 2001-2002

Knight et al., Science, 2:2, 2001.

Komberg and Baker, DNA Replication, 2nd Ed., Freeman, San Francisco, 1992.

Kuhnast et al., Bioconjug Chem, 5:627-636, 2000.

Langer et al., Proc. Natl. Acad. Sci. USA, 78(11):663-6637, 1981

Lin and Avery, Nature, 402:128-129, 1999.

Liu et al., Anal Biochem 289:239-245, 2001.

Montgomery et al., Proc. Nat'l Acad. Sci. USA, 95:155-2-15507, 1998.

Mourrain et al., Cell, 101:533, 2000.

Nicolau and Sene, Biochim. Biophys. Acta, 721:185-190, 1982.

Nicolau et al., Methods Enzymol., 149:157-176, 1987.

Nykanen et al., Cell, 107(3):309-321, 2001.

Omirulleh et al., Plant Mol Biol., 21(3):415-428, 1993.

Piutlle et al., Gene, 112(1):101-5, 1992.

Plasterk and Ketting, “The silence of the genes,” Curr. Opin. Genet. Dev., 10:562-567, 2000.

Potrykus et al., Mol. Gen. Genet., 199(2):169-77, 1985.

Regnier and Preat, Pharm Res, 10: 1596-602, 1998.

Reisfeld et al. Biochem Biophysic Res Comm, 142(2):519-526, 1987.

Richardson and Gumport, Nucleic Acids Res, 11(18):6167-84, 1983.

Richardson and Macy, Biochemistry, 20(5):1133-9, 1981.

Rippe et al., Mol. Cell Biol., 10:689-695, 1990.

Roychoudhury and Kossel, Eur J Biochem, 22(3):310-20, 1971.

Rusckowski et al., Antisense Nucleic Acid Drug Dev, 5:333-345, 2000.

Saiki et al. Science, 230:1350-1354, 1985

Sambrook et al., In: Molecular cloning, Cold Spring Harbor Laboratory Press, Cold Spring Harbor, N.Y., 1989.

Scheit, “Nucleotide Analogs,” In: Synthesis and Biological Function, Wiley-Interscience, New York, pp. 171-172, 1980.

Sedelnikova et al., Antisense Nucleic Acid Drug Dev, 6:443-452, 2000.

Sharp and Zamore, Science, 287:2431-2433, 2000.

Smardon et al., Curr Biol, 10:169-178, 2000.

Sodja et al., Nucleic Acids Res, 5(2):385-401, 1978.

Sproat et al., Nucleic Acids Research, 17(9):3373-3386, 1989.

Stalnacke et al., Eur J Nucl Med, 5:166-170, 1985.

Sui et al., Proc Natl Acad Sci USA, 99(8):5515-5520, 2002.

Tabara et al., Cell, 99:123-132, 1999.

Takeda and Ikeda, Nucl. Acids Research, 15:101-104, 1984.

Tuschl, Chembiochem, 2:239-245, 2001.

Uhlenbeck et al., Nucleic Acids Res, 10(11):3341-52, 1982.

Urdea et al., Clinical Chemistry, 35(8):1571-1575, 1989.

Viscidi et al., J Clinical Microbiology, 23(2):311-317, 1986.

Vyas et al., Crit Rev Ther Drug Carrier Syst., 18:1-76, 2001.

Waterhouse et al., Nature, 411:834-842, 2001.

Weeks et al., Clin. Chem., 29(8):1474-1479, 1983.

Williams et al., Int J Dev Biol, 41(2):3 59-364, 1997.

Winter and Brownlee, Nucleic Acids Res, 5(9):3129-39, 1978.

Wong et al., Gene, 10: 87-94, 1980.

Wu et al., Eur J Pharm Sci, 3:179-186, 2000.

Wu-Scharf et al., Science, 290:1159-1162, 2000.

Yoo et al., Nucleic Acids Res, 21:4225-4231, 2000.

Zamore, Nat. Struct. Biol. 8:746-750, 2001.

Zamore et al., Cell,.101:25-33, 2000.

Zhang et al., J Mol Neurosci. 1:13-28, 1996.

Zhang et al., Eur J Nucl Med., 11:1700-1707, 2000.

Zhang et al., J Nucl Med, 11:1660-1669,2001.

What is claimed is:

1. A method for labeling a dsRNA molecule capable of inducing RNA interference comprising:
a) hybridizing sense and antisense regions of at least one RNA strand; and,
b) labeling at least one RNA strand with a fluorescent dye.

2. The method of claim 1, wherein the sense and antisense regions are on the same strand.

3. The method of claim 1, wherein the sense and antisense regions are on separate strands.

4. The method of claim 1, wherein at least one RNA strand is labeled before the sense and antisense RNA regions are hybridized.

5. The method of claim 1, wherein the hybridized dsRNA molecule is internally labeled.

6. The method of claim 1, wherein the hybridized dsRNA molecule is labeled by incubating the molecule with a) labeling buffer comprising a physiological buffer with a pH of 7.0 to 7.5 and b) a labeling reagent comprising the fluorescent dye.

7. The method of claim 6, wherein the hybridized dsRNA molecule is end-labeled.

8. The method of claim 7, wherein end labeling comprises:
a) incubating a hybridized dsRNA molecule with a 5′ thiophosphate moiety with a thio-reactive fluorescent dye.

9. The method of claim 8, wherein the thio-reactive dye is BODIPY, Alexa Fluor, HEX, ROX, SYPRO, fluorescein, Oregon Green, tetramethylrhodamine, Texas Red, cyanine dye, or derivatives thereof

10. The method of claim 8, wherein the hybridized dsRNA molecule with a 5′ thiophosphate moiety is incubated with the thio-reactive fluorescent dye for at least 5 minutes at a temperature of 25 ° C. to 75° C.

11. The method of claim 10, wherein the hybridized dsRNA molecule with a 5′ thiophosphate moiety is incubated with the thio-reactive fluorescent dye at a temperature of 60 ° C. to 70° C.

12. The method of claim 8, wherein the hybridized dsRNA molecule with a 5′ thiophosphate moiety is created by incubating the hybridized dsRNA molecule with a kinase, a kinase buffer, and ATPγS.

13. The method of claim 1, wherein the fluorescent dye is fluorescein, Texas red, rhodamine, DAPI, Cy3, or Cy5.

14. The method of claim 1, further comprising isolating the labeled and hybridized dsRNA.

15. The method of claim 14, further comprising measuring the absorbance of the dsRNA.

16. A fluorescently labeled dsRNA molecule capable of inducing RNA interference.

17. The fluorescently labeled dsRNA molecule of claim 16, wherein two strands are labeled.

18. The fluorescently labeled dsRNA molecule of claim 16, wherein the molecule is a single strand comprising at least two regions complementary to each other.

19. The fluorescently labeled dsRNA molecule of claim 18, wherein the complementary regions include at least 5 contiguous bases.

20. The fluorescently labeled dsRNA molecule of claim 16, wherein the antisense strand is labeled.

21. The fluorescently labeled, dsRNA molecule of claim 16, wherein the sense strand is labeled.

22. The fluorescently labeled dsRNA molecule of claim 17, wherein the sense strand is labeled with a different color label than the antisense strand.

23. The fluorescently labeled dsRNA molecule of claim 16, wherein the RNA imolecule is an siRNA.

24. The fluorescently labeled, dsRNA molecule of claim 23, wherein the siRNA is 5 to 25 bases or basepairs in length.

25. The fluorescently labeled, dsRNA molecule of claim 24, wherein the siRNA is 10 to 21 bases or basepairs in length.

26. The fluorescently labeled dsRNA molecule of claim 24, wherein the siRNA is at least 10 bases or basepairs in length.

27. The fluorescently labeled dsRNA molecule of claim 16, wherein the RNA molecule is between 25 and 10,000 bases or basepairs in length.

28. The fluorescently labeled dsRNA molecule of claim 27, wherein the RNA molecule is capable of being processed into at least one siRNA in a cell.

29. The labeled dsRNA molecule of 16, wherein the label is fluorescent, enzymatic, or radioactive.

30. The labled dsRNA molecule of claim 29, wherein the label is fluorescent.

31. The fluorescently labeled dsRNA molecule of claim 30, wherein the molecule is labeled with BODIPY, Alexa Fluor, fluorescein, Oregon Green, tetramethylrhodamine, Texas Red, rhodamine, cyanine dye, or derivatives thereof

32. The fluorescently labeled dsRNA molecule of claim 16, wherein the molecule is end-labeled.

33. The fluorescently labeled dsRNA molecule of claim 16, wherein the molecule is internally labeled.

34. The fluorescently labeled dsRNA molecule of claim 16, wherein the RNA molecule comprises at least one modified residue.

35. A labeled dsRNA molecule capable of associating with an RNA-induced silencing complex.

36. A method for evaluating RNA interference in a cell comprising:
a) introducing into the cell the fluorescently labeled dsRNA molecule of claim 16; and
b) detecting or visualizing the fluorescently labeled dsRNA molecule.

37. The method of claim 36, wherein the dsRNA molecule comprises a single strand that has at least two region complementary to each other.

38. The method of claim 36, wherein the dsRNA molecule comprises two separate strands in which one strand has a sense region and the other strand has an antisense region.

39. The method of claim 36, wherein both strands of the RNA molecule are labeled.

40. The method of claim 36, wherein the antisense strand of the RNA molecule is labeled.

41. The method of claim 36, wherein the sense strand of the RNA molecule is labeled.

42. The method of claim 39, wherein the sense strand of the RNA molecule is labeled with a different color label than the antisense strand of the RNA molecule.

43. The method of claim 36, wherein the RNA molecule is an siRNA.

44. The method of claim 43, wherein the siRNA is 5 to 25 bases or basepairs in length.

45. The method of claim 44, wherein the siRNA is 10 to 21 bases or basepairs in length.

46. The method of claim 45, wherein the siRNA is at least 10 bases or basepairs in length.

47. The method of claim of claim 46, wherein the RNA molecule is between 25 and 100 bases or basepairs in length.

48. The method of claim 36, wherein the RNA molecule is capable of being processed into at least one siRNA in the cell.

49. The method of claim 36, wherein the RNA molecule is labeled with BODIPY, Alexa Fluor, fluorescein, Oregon Green, tetramethylrhodamine, Texas Red, rhodamine, cyanine dye, or derivatives thereof

50. The method of claim 36, wherein the molecule is end-labeled.

51. The method of claim 36, wherein the molecule is internally labeled.

52. The method of claim 36, wherein the cell is a eukaryotic cell.

53. The method of claim 52, wherein the cell is a mammalian cell.

54. The method of claim 53, wherein the mammalian cell is a primate, rodent, rabbit, or human cell.

55. The method of claim 36, wherein the cell is a prokaryotic cell.

56. The method of claim 36, wherein the cell is alive.

57. The method of claim 56, wherein the cell is in an organism or tissue.

58. The method of claim 36, wherein the cell is dead.

59. The method of claim 58, wherein the cell is fixed.

60. A kit for labeling a dsRNA for RNAi comprising, in a suitable container means,
a) labeling buffer comprising a physiological buffer with a pH range of 7.0 to 7.5;
b) labeling reagent for labeling dsRNA with fluorescent label comprising fluorescent dye;
c) control dsRNA comprising a dsRNA shown to trigger RNAi in a cell when labeled using components a) and b).

61. The kit of claim 60, wherein the labeling reagent further comprises an akylating agent.

62. The kit of claim 60, wherein the labeling buffer is provided at a 10× concentration.

63. The kit of claim 62, wherein the control dsRNA is single stranded.

64. The kit of claim 62, wherein the control dsRNA comprises separate complementary RNA strands.

65. The kit of claim 60, wherein the labeling reagent comprises Cy3, Cy5, or fluorescein (FAM).

66. The kit of claim 60, further comprising nuclease free water, ethanol, NaCl, Rnase inhibitor, reconstitution solution comprising DMSO, or annealing buffer comprising Hepes, potassium acetate, and magnesium acetate.
